SEQUENCE LISTING (16 sequences)

SEQ ID NO: 1 - protein, 321 aa, Escherichia coli
  PEPTIDE 1..321: note = Glucokinase
MTKYALVGDVGGTNARLALCDIASGEISQAKTYSGLDYPSLEAVIRVYLEEHKVEVKDGCIAIACPITGDWVAMTNHTWAFSIAEMKKNLGFSHLEIINDFTAVSMAIPMLKKEHLIQFGGAEPVEGKPIAVYGAGTGLGVAHLVHVDKRWVSLPGEGGHVDFAPNSEEEAIILEILRAEIGHVSAERVLSGPGLVNLYRAIVKADNRLPENLKPKDITERALADSCTDCRRALSLFCVIMGRFGGNLALNLGTFGGVFIAGGIVPRFLEFFKASGFRAAFEDKGRFKEYVHDIPVYLIVHDNPGLLGSGAHLRQTLGHIL

SEQ ID NO: 2 - protein, 456 aa, Escherichia coli
  PEPTIDE 1..456: note = Phosphomannomutase
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 3 - protein, 359 aa, Bifidobacterium longum
  PEPTIDE 1..359: note = N-acetylhexosamine-1-kinase
MTESNEDLFGIASHFALEGAVTGIEPYGDGHINTTYLVTTDGPRYILQQMNTSIFPDTVNLMRNVELVTSTLKAQGKETLDIVPTTSGATWAEIDGGAWRVYKFIEHTVSYNLVPNPDVFREAGSAFGDFQNFLSEFDASQLTETIAHFHDTPHRFEDFKAALAADKLGRAAACQPEIDFYLSHADQYAVVMDGLRDGSIPLRVTHNDTKLNNILMDATTGKARAIIDLDTIMPGSMLFDFGDSIRFGASTALEDEKDLSKVHFSTELFRAYTEGFVGELRGSITAREAELLPFSGNLLTMECGMRFLADYLEGDIYFATKYPEHNLVRTRTQIKLVQEMEQKASETRAIVADIMEAAR

SEQ ID NO: 4 - protein, 478 aa, Escherichia coli
  PEPTIDE 1..478: note = Mannose-1-phosphate guanylyltransferase
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 5 - protein, 373 aa, Escherichia coli [sequence omitted]

SEQ ID NO: 6 - protein, 321 aa, Escherichia coli
  PEPTIDE 1..321: note = GDP-L-fucose synthase
MSKQRVFIAGHRGMVGSAIKRQLEQRGDVELVLRTRDELNLLDSRAVHDFFASESIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVDYKGRVVFDASKPDGTPRKLLDVTRLHQLGWYHEISLEAGLASTYQWFLENRDRFRG

SEQ ID NO: 7 - protein, 949 aa, Bacteroides fragilis
  PEPTIDE 1..949: note = L-fucokinase
MQKLLSLPSNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAVSVFPMVNSVEELGLVLRWMLSEPELEEGKNIWLRSEHFSADEISAGANLKRLYAQREEFRKGNWKALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 8 - protein, 303 aa, Exiguobacterium acetylicum
  PEPTIDE 1..303: note = Guanosine kinase
MNKIAVIGKVFVDIKGTSFAPLHKDAKNVGDITFSNGGTGRNVAQNLAVLGNEVRFISTVTNDQIGVGVLDELKSYGANVDHVEMLEDHGMGMWLAVMDNEGDLQTSISKQPDAKLLEEAILRQSIYALDGVDAVAIDLDLSVTVLERLIHLCRKMELPLFGVCGHLSVIERNRHLLQGFTGFICSREEAEILSDLSIVTVEDAIHVANELAKKGAPFTVVTMSELGAVYVDRRTATSGHVGTKKVKVVDSTGAGDSFFSAVLSELTQEKSAEEALKLGMKVAAEVIASTENGLVPEMLDALQ

SEQ ID NO: 9 - protein, 496 aa, Pseudomonas aeruginosa
  PEPTIDE 1..496: note = 2-domain polyphosphate kinase 2
MFESAEVGHSIDKDTYEKAVIELREALLEAQFELKQQARFPVIILINGIEGAGKGETVKLLNEWMDPRLIEVQSFLRPSDEELERPPQWRFWRRLPPKGRTGIFFGNWYSQMLYARVEGHIKEAKLDQAIDAAERFERMLCDEGALLFKFWFHLSKKQLKERLKALEKDPQHSWKLSPLDWKQSEVYDRFVHYGERVLRRTSRDYAPWYVVEGADERYRALTVGRILLEGLQAALATKERAKRQPHAAPLVSSLDNRGLLDSLDLGQYLDKDAYKEQLAAEQARLAGLIRDKRFRQHSLVAVFEGNDAAGKGGAIRRVTDALDPRQYHIVPIAAPTEEERAQPYLWRFWRHIPARRQFTIFDRSWYGRVLVERIEGFCAPADWLRAYGEINDFEEQLSEYGIIVVKFWLAIDKQTQMERFKEREKTPYKRYKITEEDWRNRDKWDQYVDAVGDMVDRTSTEIAPWTLVEANDKRFARVKVLRTINDAIEAAYKKDK

SEQ ID NO: 10 - protein, 177 aa, Pasteurella multocida
  PEPTIDE 1..177: note = inorganic pyrophosphatase
MGLETVPAGKALPDDIYVVIEIPANSDPIKYEVDKESGALFVDRFMATAMFYPANYGYVNNTLSLDGDPVDVLVPTPYPLQPGSVIRCRPVGVLKMTDEAGSDAKVVAVPHSKLTKEYDHIKDVNDLPALLKAQIQHFFESYKALEAGKWVKVDGWEGVDAARQEILDSFERAKKLE

SEQ ID NO: 11 - protein, 198 aa, Sus scrofa
  PEPTIDE 1..198: note = Guanylate kinase
MGSPRPVVLSGPSGAGKSTLLKKLLQEHSSIFGFSVSHTTRDPRPGEENGKDYYFVTREVMQRDIAAGDFIEHAEFSGNLYGTSKAAVRAVQAMNRICVLDVDLQGVRNIKKTDLQPIYIFVQPPSLDVLEQRLRQRNTETEESLAKRLAAAKADMESSKEPGLFDLIIINDSLDKAYWALKEALSEEIKKAQATGHS

SEQ ID NO: 12 - protein, 558 aa, Bos taurus
  PEPTIDE 1..558: note = Glutamate dehydrogenase 1 (GLDH)
MYRYLGEALLLSRAGPAALGSASADSAALLGWARGQPAAAPQPGLVPPARRHYSEAAADREDDPNFFKMVEGFFDRGASIVEDKLVEDLKTRETEEQKRNRVRSILRIIKPCNHVLSLSFPIRRDDGSWEVIEGYRAQHSQHRTPCKGGIRYSTDVSVDEVKALASLMTYKCAVVDVPFGGAKAGVKINPKNYTDNELEKITRRFTMELAKKGFIGPGVDVPAPDMSTGEREMSWIADTYASTIGHYDINAHACVTGKPISQGGIHGRISATGRGVFHGIENFINEASYMSILGMTPGFGDKTFVVQGFGNVGLHSMRYLHRFGAKCITVGESDGSIWNPDGIDPKELEDFKLQHGTILGFPKAKIYEGSILEVDCDILIPAASEKQLTKSNAPRVKAKIIAEGANGPTTPEADKIFLERNIMVIPDLYLNAGGVTVSYFEWLNNLNHVSYGRLTFKYERDSNYHLLMSVQESLERKFGKHGGTIPIVPTAEFQDRISGASEKDIVHSGLAYTMERSARQIMRTAMKYNLGLDLRTAAYVNAIEKVFRVYNEAGVTFT

SEQ ID NO: 13 - protein, 486 aa, Leuconostoc mesenteroides
  PEPTIDE 1..486: note = Glucose-6-phosphate 1-dehydrogenase (G6PDH)
MVSEIKTLVTFFGGTGDLAKRKLYPSVFNLYKKGYLQEHFAIVGTARQQLSDEEFKQLVRDSIKDFTEDQAQAEAFIAHFSYRAHDVTDAASYGILKSAIEEAATKFDIDGNRIFYMSVAPRFFGTIAKYLKSEGLLADTGYNRLMIEKPFGTSYATAEELQSDLENAFDDDQLFRIDHYLGKEMVQNIAALRFGNPIFDAAWNKDYIKNVQVTLSEVLGVEERAGYYDTTGALLDMIQNHTMQIVGWLAMEKPESFNDKDIRAAKNAAFNALKIYNEEEVNKYFVRAQYGAGDTADYKPYLEEADVPADSKNNTFIAGELQFDLPRWEGVPFYVRSGKRLAAKQTRVDIVFKAGTFNFGSEQEAQESVLSIIIDPKGAIELKLNAKSVEDAFNTRTINLDWAVSDEDKKNTPEPYERMIHDTMNGDGSNFADWNGVSIAWKFVDAITAVYDADKAPLETYKSGSMGPEASDKLLAENGDAWVFKG

SEQ ID NO: 14 - protein, 301 aa, Ruegeria pomerovi
  PEPTIDE 1..301: note = phosphotransferase 3 (PPK3)
MNRNGSTKDPRRMTGAATGEISRYFNDKAPKDIRRAIEKADKDDILSTTYPYDAEMTAKDYRAQMEALQIELVKLQAWIKQSGARVALLFEGRDAAGKGGTIKRFRENLNPRGARVVALSKPTEAERSQWYFQRYIQHLPSAGELVFYDRSWYNRGVVEHVFGWCDEEQRERFFRQVMPFEHDLVDDGIHLFKFWLNVGRAEQLRRFHDRERDPLKQWKLSPVDIAGLDKWEAYTTAISQTLTRSHSDRAPWTVIRSDDKKRARLAAIRTVLSGIDYDNKDRAAVGQPDAAICGGPDIWDA

SEQ ID NO: 15 - protein, 359 aa, Picrophilus torridus
  PEPTIDE 1..359: note = Glucose/galactose 1-dehydrogenase (GDH)
MVRAIITNAPNGGVKIENVNINEPEHYEVKLRPVYTGLCGTDRGEVLGNLSFAYNEPGYNYLVLGHEAICQVIEASENPYKIKPGDYVVPVVRRPGKCVNCRIGREDDCSDGDKHEAGITGLHGFMRDYFYDEAKNLVKINDKNMVKVAVLTEPTKNVMKAFEVFDTVSKRSIFQGDDSTNLTKNCLIIGTGSEAFLYAFMAREYRFNVFMTNRHPVGEEKLSIISRINADFYDYTREDPLKGIDLLIDTSGDPGTIFRFVRKMNYNGVVILFGTNGRAPATSIDGEDIDYIIERNISLVGSVDGAKRHYLRAVEYLEKWNYSEGSVINRLITGVFEPEDVSIFTKKPENEIKSVIKWS

SEQ ID NO: 16 - protein, 462 aa, Helicobacter pylori
  PEPTIDE 1..462: note = alpha-1,3/4-fucosyltransferase (3/4FT)
MFQPLLDAFIDSTHLDETTHKPPLNVALANWWPLKNSEKKGFRDFILHFILKQRYKIILHSNPNEPSDLVFGNPLEQARKILSYQNTKRVFYTGENEVPNFNLFDYAIGFDELDFNDRYLRMPLYYAYLHYKAMLVNDTTSPYKLKALYTLKKPSHKFKENHPNLCALIHNESDPWKRGFASFVASNPNAPIRNAFYDALNAIEPVASGGSVKNTLGYKVKNKNEFLSQYKFNLCFENSQGYGYVTEKILDAYFSHTIPIYWGSPSVAKDFNPKSFVNVHDFNNFDEAIDYIRYLHAHQNAYLDMLYENPLNTIDGKAGFYQDLSFEKILDFFKNILENDTIYHCNDAHYSALHRDLNEPLVSVDDLRRDHDDLRVNYDDLRVNYDDLRVNYDDLRVNYDDLRVNYDDLRRDHDDLRRDHERLLSKATPLLELSQNTSFKIYRKAYQKSLPLLRAIRRWVRK

(19) United States
(12) Patent Application Publication
(10) Pub. No.: US 2024/0124911 A1
(43) Pub. Date: Apr. 18, 2024

(54) ENZYMATIC METHOD FOR PREPARATION OF GDP-FUCOSE

(71) Applicant: Max-Planck-Gesellschaft zur Förderung der Wissenschaften e.V., Munich (DE)

(72) Inventors: Reza Mahour, Magdeburg (DE); Thomas F.T. Rexer, Magdeburg (DE)

(21) Appl. No.: 18/469,401

(22) Filed: Sep. 18, 2023

Related U.S. Application Data
(62) Division of application No. 17/593,929, filed on Sep. 28, 2021, now Pat. No. 11,795,486.

(30) Foreign Application Priority Data
Apr. 1, 2019 (EP) 19166549.6

(51) Int. Cl. C12P 19/44

(52) U.S. Cl. CPC C12P 19/44

(57) ABSTRACT

The present invention relates to an enzyme-catalyzed process for producing GDP-fucose from low-cost substrates guanosine and L-fucose or guanosine and D-Mannose in a single reaction mixture. Said process can be operated (semi)continuously or in batch mode. Further, said process can be adapted to produce fucosylated molecules and biomolecules including glycans, such as human milk oligosaccharides, proteins, peptides, glycoproteins or glycopeptides.

### FIELD OF THE INVENTION

The present invention relates to an enzyme-catalyzed process for producing GDP-fucose from low-cost substrates guanosine and L-fucose or guanosine and D-mannose in a single reaction mixture. Said process can be operated semi-continuously, even continuously or in a fed batch mode. Further, said process can be adapted to produce fucosylated molecules and biomolecules including glycans, such as human milk oligosaccharides, proteins, peptides, glycoproteins or glycopeptides.

### BACKGROUND OF THE INVENTION

Guanosine 5′-diphospho-β-L-fucose (GDP-fucose or GDP-Fuc) is a key substrate for a large number of biotechnological applications and food technology. It is the substrate for the core fucosylation of anti-inflammatory antibodies which are used to treat autoimmune diseases. Moreover, GDP-fucose is needed for the production of carbohydrate vaccines and in the growing field of personalized medicine, i.e. preparation of glyconanomaterials for drug delivery. In infant food (human milk), fucosylated oligosaccharides comprise the majority of sugars and, thus, there is a high demand to include fucosylated sugars in synthetically produced dairy products for infants.

However, in spite of the high demand for GDP-fucose (in the order of tons per year), the availability of GDP-fucose is very limited, even for researchers. Up to now, the price of low endotoxin GDP-fucose is above 3,000 Euros per gram. Due to the high price of GDP-fucose not only basic and applied research activities are hampered but also industrial applications are hindered.

Bioprocess engineering strategies to synthesize GDP-fucose can be classified into in vivo and in vitro processes: Microorganisms are metabolically engineered in order to produce GDP-fucose, either intracellularly or extracellularly, as part of their metabolism. However, low yields, high levels of unwanted by-products, the required time for cell line design and the complicated scale up are drawbacks. Taking into account regulatory aspects, specifically for infant food, application of genetically modified organisms (GMOs) can severely delay the approval process.

GDP-fucose can also be produced in vitro by using biocatalytic (enzymatic) processes (see APMIS 2006, 114, 539-548). For example, the enzymatic synthesis of GDP-fucose from L-fucose and guanosine triphosphate using bifunctional enzyme L-fucose pyrophosphorylase (FKP) is reported by Zhao et al. (Nat Protoc. 2010 5(4): 636-646). Wittmann et al. (J. Org. Chem. 1997, 62, 2144-2147) describe the synthesis of GDP-fucose from fucose-1-phosphate and guanosine 5′-monophosphomorpholidate using phosphoamidite chemistry. Tonetti et al. (J. Biol. Chem. 1996, 271(44), 27274-27279) report on the homodimeric NADP(H)-binding protein FX, which apparently catalyzes a combined epimerase and NADPH-dependent reductase activity, thus converting GDP-4-keto-6-D-deoxymannose to GDP-L-fucose. Sullivan et al. (J. Biol. Chem. 1998, 273(14), 8193-8202) describe the in vitro preparation of GDP-fucose from GDP-mannose using recombinant human GDP-mannose4,6-dehydratase and the FX protein. Lau et al. report on the biosynthesis of GDP-L-fucose from GDP-D-mannose and investigated the enzyme GDP-fucose synthase which is involved in the synthesis. The enzyme converts GDP-4-keto-6-deoxy-D-mannose into GDP-L-fucose (J. Am. Chem. Soc. 2008, 130, 17593-17602). Rexer et al. report one-pot synthesis of GDP-mannose from mannose by a multi-enzyme cascade of glucokinase (GIk), phosphomannomutase (ManB), mannose-1-phosphate-guanylyltransferase (ManC), inorganic pyrophosphatase (PmPpA), and 1-domain polyphosphate kinase 2 (1 D-Ppk2) expressed in E. coli. (Biotechnology and Bioengineering 2018, 115, 192-205). There is a long-felt need for a method of producing GDP-fucose in a cost-effective manner starting from low cost and readily available substrates.

Thus, it is the objective of the present invention to provide a cost-effective and efficient method for the preparation of GDP-fucose.

The objective of the present invention is solved by the teaching of the independent claims. Further advantageous features, aspects and details of the invention are evident from the dependent claims, the description, the figures, and the examples of the present application.

### DESCRIPTION OF THE INVENTION

In biochemistry nucleotide sugars are well known as active forms of monosaccharides and in glycosylation reactions nucleotide sugars are known to act as glycosyl donors. Glycosyltransferases (GTFs) are enzymes that catalyze the transfer of saccharide moieties from activated nucleotide sugars to nucleophilic glycosyl acceptor molecules. Thus, in biochemistry the glycosylation reactions are catalyzed by glycosyltransferases.

In order to act as glycosyl donors it is essential that the respective monosaccharides are present in a highly energetic form, like for example in form of nucleotide sugars, particularly nucleotide diphospho sugars derived from uridine diphosphate, guanosine diphosphate or cytosine diphosphate and so on. Examples of well known nucleotide sugars are UDP-glucose, UDP-galactose, UDP-GlcNAc, UDP-GalNac, UDP-xylose, UDP-glucuronic acid, GDP-mannose and GDP-fucose. It is well known that the conversion of simple monosaccharides into activated nucleotide sugars can be achieved by enzyme catalyzed reaction of a nucleoside triphosphate (NTP) and a glycosyl monophosphate, wherein the glycosyl monophosphate contains a phosphate group at the anomeric carbon.

In order to obtain a nucleoside diphosphate (NDP)-monosaccharide the used monosaccharide needs to be converted into a glycosyl monophosphate derivative. In general, said reaction can be accomplished by applying specific enzymes like phosphotransferases and additionally phosphomutases, if required, to obtain the desired monosaccharide-1-phosphate. Phosphotransferases are enzymes classified under EC number 2.7 that catalyze phosphorylation reactions. Phosphotransferases are further classified according to their acceptor molecule. For example, phosphotransferases under EC 2.7.1 are phosphotransferases with an alcohol group as acceptor. Phosphomutases are isomerases, i.e. enzymes that can catalyze an internal transfer of a phosphate group. Phosphomutases are required in case the phosphorylation of the substrate via phosphotransferase results in a monosaccharide-6-phosphate, like in case of D-mannose or D-glucose for example mannose-6-phosphat or glucose-6-phosphat respectively. The respective phosphomutase then catalyzes the internal transfer of the phosphate group which results in the conversion of mannose-6-phosphate into mannose-1-phosphate or glucose-6-phosphate into glucose-1-phosphate, respectively.

Kinases are enzymes which form a part of the family of the phosphotransferases. Kinases are enzymes that catalyze the transfer of phosphate groups from high-energy, phosphate-donating molecules to specific substrates. This process is known as phosphorylation, where the substrate gains a phosphate group and the high-energy adenosine triphosphate (ATP) molecule donates a phosphate group. This transesterification produces a phosphorylated substrate and ADP. Thus, in order to obtain a monosaccharide-1-phosphate, suitable kinases like a fucokinase or N-acetylhexosamine-1-kinase may be applied to obtain fucose-1-phosphate from L-fucose or mannose-1-phosphate from D-mannose respectively.

With the use of nucleotidyltransferases a nucleoside triphosphate (NTP) and a monosaccharide-1-phosphate can be converted to the respective nucleoside diphosphate (NDP)-monosaccharide. Nucleotidyltransferases are transferase enzymes of phosphorus-containing groups and are classified under EC number 2.7.7. For the different naturally occurring nucleotides nucleotide-specific nucleotidyltransferases are known in the art, e.g. uridyltransferases transfer uridylyl-groups, adenyltransferases transfer adenylyl-groups, guanylyltransferases transfer guanylyl-groups, cytidylyltransferases transfer cytidylyl-groups and thymidilyl-transferases transfer thymidilyl-groups. Thus, nucleotidyltransferases are suitable to catalyze the reaction of monosaccharide-1-phosphates with nucleoside triphosphates, e.g. fucose-1-phosphate with guanosine triphosphate (GTP) to obtain GDP-fucose or mannose-1-phosphate with guanosine triphosphate (GTP) to obtain GDP-mannose. In case of GDP-fucose and GDP-mannose a guanylyltransferase is suitable for catalyzing the reaction with guanosine triphosphate (GTP).

Guanosine diphosphate (GDP)-monosaccharides which relate to naturally occurring GDP-monosaccharides are GDP-mannose and GDP-fucose. The above described general reaction scheme to obtain the GDP-monosaccharides can be conducted with guanosine triphosphate and mannose-1-phosphat (Man-1-P) in case of GDP-mannose and fucose-1-phosphate (Fuc-1-P) in case of GDP-fucose with specific guanylyltransferases which catalyze the reaction to obtain the desired GDP-mannose or GDP-fucose respectively.

However one disadvantage of the enzyme-catalyzed reaction scheme to obtain nucleoside diphosphate (NDP)-monosaccharides is based on the fact that the starting materials, in particular the respective nucleoside triphosphates are very expensive and thus the synthesis pathway results in a cost-intensive synthesis of NDP-monosaccharides and in particular of GDP-fucose or GDP-mannose. As already described above for GDP-fucose there is a need in the art to provide a cost effective and efficient method for preparation of nucleoside diphosphate monosaccharides, like GDP-fucose or GDP-mannose, and in particular there is a need to provide a cost effective and efficient method for preparation of GDP-fucose from low cost and readily available starting materials.

With regard to GDP-monosaccharides, GDP-fucose and GDP-mannose relate to naturally occurring activated GDP-sugars in mammals. Therefore guanosine has been identified as suitable nucleotide and L-fucose and D-mannose have been identified as suitable monosaccharides for the preparation of GDP-fucose. It should be clear that with regard to an enzyme-catalyzed reaction at least suitable enzymes must be provided. Therefore the inventors have identified guanosine and readily available monosaccharides, such as L-fucose or D-mannose as suitable starting materials for the production of GDP-fucose in an enzymatic one-pot cascade reaction.

A process for biocatalytic production of GDP-fucose starting from guanosine and L-fucose or guanosine and D-mannose was not established so far.

In order to provide a cost-effective and efficient method for the preparation of GDP-fucose guanosine and L-fucose were identified as suitable starting materials for the production of GDP-fucose in an enzymatic cascade reaction as depicted in FIG. 1 which consists of (a) the formation of fucose-1-phosphate (Fuc-1-P) from L-fucose and adenosine triphosphate (ATP; catalytic amount), (b) the formation of guanosine triphosphate (GTP) from guanosine and polyphosphate, and (c) the reaction of fucose-1-phosphate with guanosine triphosphate (GTP) to GDP-fucose. It was envisioned that GDP-fucose may be produced from L-fucose and guanosine in the presence of a guanosine kinase, a polyphosphate kinase, and a L-fucokinase/L-fucose-1-phosphate guanylyltransferase in a suitable buffer (FIG. 2).

Furthermore guanosine and D-mannose were identified as suitable starting materials for the production of GDP-fucose in an enzymatic cascade reaction as depicted in FIG. 3 which consist of (a) the formation of mannose-1-phosphat (Man-1-P) from D-mannose and adenosine triphosphate (ATP), (b) the formation of guanosine triphosphate (GTP) from guanosine and polyphosphate, and (c) the reaction of mannose-1-phosphate with guanosine triphosphate (GTP) to GDP-mannose and (d) the conversion of GDP-mannose to GDP-fucose. It was envisioned that GDP-fucose may be produced from D-mannose and guanosine in the presence of a guanosine kinase, a polyphosphate kinase and either a glucokinase, a phosphomannomutase, a mannose-1-phosphate guanylyltransferase, a GDP-mannose 4,6-dehydratase and a GDP-L-fucose synthase or a N-acetylhexosamine-1-kinase, a mannose-1-phosphate guanylyltransferase, a GDP-mannose-4,6-dehydratase and a GDP-L-fucose-synthase in a suitable buffer.

However, any attempts to produce GDP-fucose from L-fucose and guanosine or D-mannose and guanosine in this enzymatic cascade reaction were unsuccessful and no formation of GDP-fucose was observed (Example 3).

Surprisingly, the inventors have found that by solubilizing guanosine in a co-solvent, such as dimethyl sulfoxide, a nearly complete conversion of guanosine and L-fucose to GDP-fucose was achieved after already three hours (Example 2). Also, the co-solvent did not affect the activity of the enzymes used in the preparation of GDP-fucose. The titer is above the solubility of the substrate, which is achieved through using a co-solvent. Furthermore, the inventors have found that by solubilizing guanosine in a co-solvent, such as dimethyl sulfoxide, a nearly complete conversion of guanosine and D-mannose to GDP-fucose was achieved. Also, the co-solvent did not affect the activity of the enzymes used in the preparation of GDP-fucose from guanosine and D-mannose.

For example, the method of the present invention is beneficial over the above described methods known in the art for the enzymatic synthesis of GDP-fucose from L-fucose and guanosine triphosphate, since the expensive guanosine triphosphate starting material can be avoided and replaced with simpler nucleoside guanosine, which results in a cost-effective and efficient method for the preparation of GDP-fucose, as described herein.

Thus, the present invention is directed to a method for producing guanosine 5′-diphospho-β-L-fucose from guanosine and L-fucose or from guanosine and D-mannose comprising the following steps:

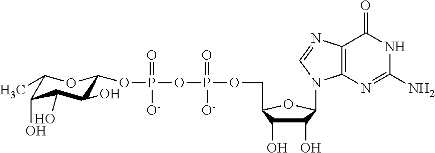

A) providing a solution comprising
   (i) guanosine and L-fucose or guanosine and D-mannose represented by the following formulae

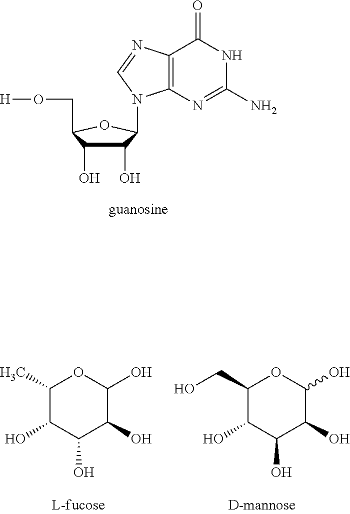

(ii) polyphosphate, adenosine triphosphate and a co-solvent for solubilizing guanosine; and
providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent.

The present invention is directed to a method for producing guanosine 5′-diphospho-β-L-fucose from guanosine and L-fucose or from guanosine and D-mannose comprising the following steps:

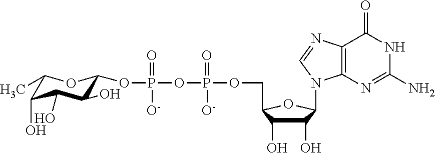

A) providing a solution comprising
   (i) guanosine and L-fucose or guanosine and D-mannose represented by the following formulae

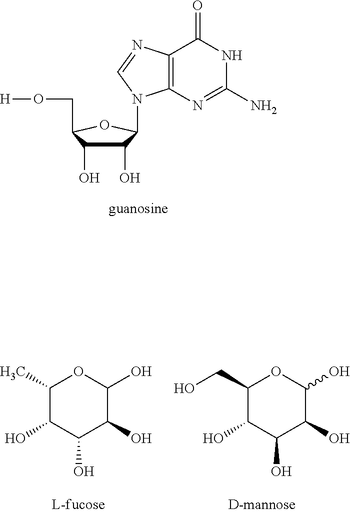

(ii) polyphosphate, adenosine triphosphate, a co-solvent for solubilizing guanosine and in case of D-mannose NADPH; and
providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate, NADPH and the co-solvent.

In a preferred embodiment the present invention is directed to a method for producing guanosine 5′-diphospho-β-L-fucose from guanosine and D-mannose comprising the following steps:

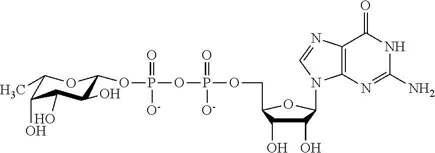

A) providing a solution comprising
   (i) guanosine and D-mannose represented by the following formulae

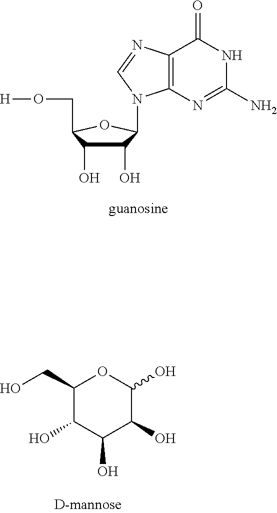

(ii) polyphosphate, adenosine triphosphate and a co-solvent for solubilizing guanosine; and
providing a set of enzymes comprising either (a) a guanosine kinase, a polyphosphate kinase and a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent.

In a preferred embodiment the present invention is directed to a method for producing guanosine 5′-diphospho-β-L-fucose from guanosine and L-fucose comprising the following steps:

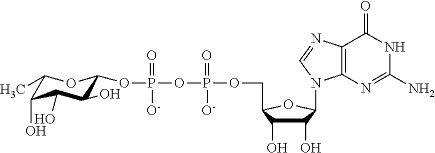

A) providing a solution comprising
   (i) guanosine and L-fucose represented by the following formulae

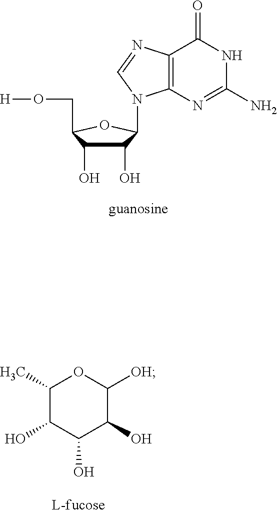

(ii) polyphosphate, adenosine triphosphate and a co-solvent for solubilizing guanosine; and
providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent.

Alternatively worded, the present invention is directed to a method for producing guanosine 5′-diphospho-β-L-fucose from guanosine and L-fucose or D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent.

Alternatively worded, the present invention is directed to a method for producing guanosine 5′-diphospho-β-L-fucose from guanosine and L-fucose comprising the following steps:
A) providing a solution comprising
   (i) guanosine and L-fucose;
   (ii) polyphosphate, adenosine triphosphate and a co-solvent for solubilizing guanosine; and
   providing the following enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the enzymes, polyphosphate, adenosine triphosphate and the co-solvent.

Also, the present invention is directed to a method for producing guanosine 5′-diphospho-β-L-fucose from guanosine and L-fucose comprising the following steps:
A) providing a solution comprising
   (i) guanosine and L-fucose;
   (ii) polyphosphate, adenosine triphosphate and a co-solvent for solubilizing guanosine; and
   providing the following enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the enzymes, polyphosphate, adenosine triphosphate and the co-solvent,
wherein the conversion of guanosine to guanosine 5′-diphospho-β-L-fucose is at least 78% after 3 hours.

The production step B) of guanosine 5′-diphospho-β-L-fucose from guanosine and L-fucose according to the invention comprises
(a) forming fucose-1-phosphate (Fuc-1-P) from L-fucose and adenosine triphosphate being catalyzed by a L-fucokinase/L-fucose-1-phosphate guanylyltransferase,
(b) forming guanosine triphosphate (GTP) from guanosine, adenosine triphosphate and polyphosphate being catalyzed by a guanosine kinase and a polyphosphate kinase; and
(c) reacting fucose-1-phosphate with guanosine triphosphate to GDP-fucose in the presence of a L-fucokinase/L-fucose-1-phosphate guanylyltransferase.

Apparently, the steps (a) and (b) may be carried out simultaneously or successively. Also, their order may be reverted to (b)→(a)→(c).

Thus, the present invention is directed to a method for producing guanosine 5′-diphospho-β-L-fucose from guanosine and L-fucose comprising the following steps:
A) providing a solution comprising
   (i) guanosine and L-fucose;
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent by
   (a) forming fucose-1-phosphate from L-fucose and adenosine triphosphate being catalyzed by a L-fucokinase/L-fucose-1-phosphate guanylyltransferase,
   (b) forming guanosine triphosphate from guanosine, adenosine triphosphate and polyphosphate being catalyzed by a guanosine kinase and a polyphosphate kinase; and
   (c) reacting fucose-1-phosphate with guanosine triphosphate to GDP-fucose in the presence of a L-fucokinase/L-fucose-1-phosphate guanylyltransferase.

More specifically, the production step B) of guanosine 5′-diphospho-β-L-fucose from guanosine and fucose according to the invention comprises
(a) forming fucose-1-phosphate (Fuc-1-P) from L-fucose and adenosine triphosphate being catalyzed by a L-fucokinase/L-fucose-1-phosphate guanylyltransferase,
(b1) forming guanosine monophosphate (GMP) from guanosine and adenosine triphosphate being catalyzed by a guanosine kinase;
(b2) forming guanosine triphosphate (GTP) from guanosine monophosphate and polyphosphate being catalyzed by a polyphosphate kinase; and
(c) reacting fucose-1-phosphate with guanosine triphosphate to GDP-fucose in the presence of a L-fucokinase/L-fucose-1-phosphate guanylyltransferase.

Apparently, the step (a) may be carried out before, simultaneously to or after step (b1) or (b2). Thus, the step order may also be reverted to (b1)→(b2)→(a)→(c).

Thus, the present invention is directed to a method for producing guanosine 5′-diphospho-β-L-fucose from guanosine and L-fucose comprising the following steps:
A) providing a solution comprising
   (i) guanosine and L-fucose;
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and L-fucose in the presence of the set of enzymes, polyphosphate, adenosine triphosphate and the co-solvent by
   (a) forming fucose-1-phosphate from L-fucose and adenosine triphosphate being catalyzed by a L-fucokinase/L-fucose-1-phosphate guanylyl-transferase,
   (b1) forming guanosine monophosphate from guanosine and adenosine triphosphate being catalyzed by a guanosine kinase;
   (b2) forming guanosine triphosphate from guanosine monophosphate and polyphosphate being catalyzed by a polyphosphate kinase; and
   (c) reacting fucose-1-phosphate with guanosine triphosphate to GDP-fucose in the presence of a L-fucokinase/L-fucose-1-phosphate guanylyltransferase.

Even more specifically, the production step B) of guanosine 5′-diphospho-β-L-fucose from guanosine and L-fucose according to the invention comprises
(a) forming fucose-1-phosphate (Fuc-1-P) from L-fucose and adenosine triphosphate being catalyzed by a L-fucokinase/L-fucose-1-phosphate guanylyltransferase,
(b1) forming guanosine monophosphate (GMP) from guanosine and adenosine triphosphate being catalyzed by a guanosine kinase;
(b2′) forming guanosine diphosphate (GDP) from guanosine monophosphate and polyphosphate being catalyzed by a polyphosphate kinase
(b2″) forming guanosine triphosphate (GTP) from guanosine diphosphate and polyphosphate being catalyzed by a polyphosphate kinase; and
(c) reacting fucose-1-phosphate with guanosine triphosphate to GDP-fucose in the presence of a L-fucokinase/L-fucose-1-phosphate guanylyltransferase.

Apparently, the step (a) am be carried out before, simultaneously to or after steps (b1), (b2′) and (b2″). Thus, the step order may also be reverted to (b1)→(b2′)→(b2″)→(a)→(c).

Thus, the present invention is directed to a method for producing guanosine 5′-diphospho-β-L-fucose from guanosine and L-fucose comprising the following steps:
A) providing a solution comprising
   (i) guanosine and L-fucose;
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and L-fucose in the presence of the set of enzymes, polyphosphate, adenosine triphosphate and the co-solvent by
   (a) forming fucose-1-phosphate from L-fucose and adenosine triphosphate being catalyzed by a L-fucokinase/L-fucose-1-phosphate guanylyl-transferase,
   (b1) forming guanosine monophosphate from guanosine and adenosine triphosphate being catalyzed by a guanosine kinase;
   (b2′) forming guanosine diphosphate from guanosine monophosphate and polyphosphate being catalyzed by a polyphosphate kinase
   (b2″) forming guanosine triphosphate from guanosine diphosphate and polyphosphate being catalyzed by a polyphosphate kinase; and
   (c) reacting fucose-1-phosphate with guanosine triphosphate to GDP-fucose in the presence of a L-fucokinase/L-fucose-1-phosphate guanylyltransferase.

The production step B) of guanosine 5′-diphospho-α-D-mannose from guanosine and D-mannose according to the invention comprises
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase
(b) forming guanosine triphosphate (GTP) from guanosine, adenosine triphosphate and polyphosphate being catalyzed by a guanosine kinase and a polyphosphate kinase; and
(c) reacting mannose-1-phosphate with guanosine triphosphate to GDP-mannose in the presence of a D-mannose-1-phosphate guanylyltransferase.

The production step B) of guanosine 5′-diphospho-β-L-fucose from guanosine and D-mannose according to the invention comprises
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase
(b) forming guanosine triphosphate (GTP) from guanosine, adenosine triphosphate and polyphosphate being catalyzed by a guanosine kinase and a polyphosphate kinase; and
(c) reacting mannose-1-phosphate with guanosine triphosphate to GDP-mannose in the presence of a D-mannose-1-phosphate guanylyltransferase
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

Apparently, the steps (a) and (b) may be carried out simultaneously or successively. Also, their order may be reverted to (b)→(a)→(c).

Thus, the present invention is directed to a method for producing guanosine 5′-diphospho-β-L-fucose from guanosine and D-mannose comprising the following steps:
A) providing a solution comprising
   (i) guanosine and D-mannose;
   (ii) polyphosphate, adenosine triphosphate and a co-solvent for solubilizing guanosine; and
   providing a set of enzymes comprising a guanosine kinase, a polyphosphate kinase and either (a) a glucokinase, phosphomannomutase, a mannose-1-phosphate guanylyltransferase, a GDP-mannose-4,6-dehydratase and a GDP-L-fucose-synthase or (b) a N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent by
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase
(b) forming guanosine triphosphate (GTP) from guanosine, adenosine triphosphate and polyphosphate being catalyzed by a guanosine kinase and a polyphosphate kinase; and
(c) reacting mannose-1-phosphate with guanosine triphosphate to GDP-mannose in the presence of a D-mannose-1-phosphate guanylyltransferase
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

More specifically, the production step B) of guanosine 5′-diphospho-β-L-fucose from guanosine and D-mannose according to the invention comprises
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase
(b1) forming guanosine monophosphate (GMP) from guanosine and adenosine triphosphate being catalyzed by a guanosine kinase;
(b2) forming guanosine triphosphate (GTP) from guanosine monophosphate and polyphosphate being catalyzed by a polyphosphate kinase; and
(c) reacting mannose-1-phosphate with guanosine triphosphate to GDP-mannose in the presence of a D-mannose-1-phosphate guanylyltransferase
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

Apparently, the step (a) may be carried out before, simultaneously to or after step (b1) or (b2). Thus, the step order may also be reverted to (b1)→(b2)→(a)→(c). Thus, the present invention is directed to a method for producing guanosine 5′-diphospho-β-L-fucose from guanosine and D-mannose comprising the following steps:
A) providing a solution comprising
   (i) guanosine and D-mannose;
   (ii) polyphosphate, adenosine triphosphate and a co-solvent for solubilizing guanosine; and
   providing a set of enzymes comprising a guanosine kinase, a polyphosphate kinase and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent by
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase
(b1) forming guanosine monophosphate (GMP) from guanosine and adenosine triphosphate being catalyzed by a guanosine kinase;
(b2) forming guanosine triphosphate (GTP) from guanosine monophosphate and polyphosphate being catalyzed by a polyphosphate kinase; and
(c) reacting mannose-1-phosphate with guanosine triphosphate to GDP-mannose in the presence of a D-mannose-1-phosphate guanylyltransferase
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

Even more specifically, the production step B) of guanosine 5′-diphospho-β-L-fucose from guanosine and D-mannose according to the invention comprises
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomanno-mutase
(b1) forming guanosine monophosphate (GMP) from guanosine and adenosine triphosphate being catalyzed by a guanosine kinase;
(b2′) forming guanosine diphosphate (GDP) from guanosine monophosphate and polyphosphate being catalyzed by a polyphosphate kinase
(b2″) forming guanosine triphosphate (GTP) from guanosine diphosphate and polyphosphate being catalyzed by a polyphosphate kinase; and
(c) reacting mannose-1-phosphate with guanosine triphosphate to GDP-mannose in the presence of a D-mannose-1-phosphate guanylyltransferase
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

Apparently, the step (a) am be carried out before, simultaneously to or after steps (b1), (b2′) and (b2″). Thus, the step order may also be reverted to (b1)→(b2′)→(b2″)→(a)→(c).

Thus, the present invention is directed to a method for producing guanosine 5′-diphospho-β-L-fucose from guanosine and D-mannose comprising the following steps:
A) providing a solution comprising
   (i) guanosine and D-mannose;
   (ii) polyphosphate, adenosine triphosphate and a co-solvent for solubilizing guanosine; and
   providing a set of enzymes comprising a guanosine kinase, a polyphosphate kinase and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent by
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase
(b1) forming guanosine monophosphate (GMP) from guanosine and adenosine triphosphate being catalyzed by a guanosine kinase;
(b2′) forming guanosine diphosphate (GDP) from guanosine monophosphate and polyphosphate being catalyzed by a polyphosphate kinase
(b2″) forming guanosine triphosphate (GTP) from guanosine diphosphate and polyphosphate being catalyzed by a polyphosphate kinase; and
(c) reacting mannose-1-phosphate with guanosine triphosphate to GDP-mannose in the presence of a D-mannose-1-phosphate guanylyltransferase
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

The inventive method for producing GDP-fucose has the following significant advantages over the methods described in the prior art:
significant cost reduction with respect to starting materials, i.e. no expensive GDP or GTP is required,
the method can be performed in a continuous manner, thereby potentially allowing providing GDP-fucose on a ton scale per year,
cell-free process, thereby avoiding adverse GMO aspects (regulation, labelling),
direct use of cell-free extracts, no costs for biocatalyst purification,
enzymes can be immobilized on low-cost, commercially available and ready to use solid supports,
nearly quantitative yield with respect to guanosine,
high scalability renders the inventive method useful for industrial applications.

Due to the addition of small amounts of a co-solvent for solubilizing guanosine, the inventors were able to establish a multi enzyme cascade reaction from guanosine to guanosine 5′-diphospho-β-L-fucose with a high conversion rate. Preferably, the co-solvent is an organic solvent selected from the group comprising: methanol, ethanol, isopropanol, n-propanol, isobutanol, n-butanol, tert-butanol, acetonitrile, acetone and dimethyl sulfoxide. Preferably the co-solvent is a polar aprotic solvent, such as dimethyl sulfoxide or dimethylformamide. Preferably the co-solvent does not inhibit enzyme activity. More preferably, the co-solvent is dimethyl sulfoxide.

Thus, the present invention is directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,
wherein the co-solvent for solubilizing guanosine is dimethyl sulfoxide.

Thus, the present invention is directed to a method for producing guanosine 5′-diphospho-β-L-fucose from guanosine and D-mannose comprising
A) providing a solution comprising
   (i) guanosine and D-mannose;
   (ii) polyphosphate, adenosine triphosphate and a co-solvent for solubilizing guanosine; and
   providing a set of enzymes comprising a guanosine kinase, a polyphosphate kinase and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent by
wherein the co-solvent for solubilizing guanosine is dimethyl sulfoxide.

### DETAILED DESCRIPTION OF THE INVENTION

### Definitions

As used herein, the term “polyphosphate” refers to any salts containing several P—O—P bonds generated by corner sharing of six or more phosphate (PO₄) tetrahedral, leading to the formation of long chains. The term “PolyPₙ” is synonymously used, wherein n represents average chain length of the number of phosphate residues, e.g. PolyP₂₅ refers to a polyphosphate having about 25 phosphate residues and PolyP₁₄ refers to a polyphosphate having about 14 phosphate residues.

As used herein, the term “guanosine kinase” or “inosine kinase” refers to a polypeptide having guanosine kinase activity, i.e. a guanosine kinase catalyzes the reaction of guanosine to guanosine 5′-monophosphate in the presence of adenosine triphosphate. The guanosine kinase belongs to the EC class 2.7.1.73.

As used herein, the term “polyphosphate kinase” refers to a polypeptide having polyphosphate kinase activity, i.e. a polyphosphate kinase catalyzes the following reactions:

ADP+polyphosphate(n+1) [symbol] ATP+polyphosphate(n)

NMP+polyphosphate(n+1) [symbol] NDP+polyphosphate(n)

NDP+polyphosphate(n+1) [symbol] NTP+polyphosphate(n)

with N being a nucleotide such as guanosine, adenosine, uridine etc. and NMP being nucleoside monophosphate, NDP being nucleoside diphosphate and NTP being nucleoside triphosphate.

In case of guanosine the polyphosphate kinase catalyzes the following reactions:

ADP+polyphosphate(n+1) [symbol] ATP+polyphosphate(n)

GMP+polyphosphate(n+1) [symbol] GDP+polyphosphate(n)

GDP+polyphosphate(n+1) [symbol] GTP+polyphosphate(n)

The polyphosphate kinase belongs to the EC class 2.7.4.1. Representatives of the polyphosphate kinase enzyme used in the inventive methods described herein include but are not limited to polyphosphate kinase 1 (PPK1), polyphosphate kinase 2 (PPK2), 2-domain polyphosphate kinase 2 (2D-PPK2) 1-domain polyphosphate kinase 2 (1D-PPK2), polyphosphate kinase 3 (PPK3) and guanylate kinase (EC class 2.7.4.8).

As used herein, the term “L-fucokinase/L-fucose-1-phosphate guanylyl-transferase” refers to a bifunctional polypeptide having L-fucokinase activity and L-fucose-1-phosphate guanylyltransferase activity, i.e. a polypeptide that catalyzes the following reactions:

[symbol]

Fuc+ATP [symbol] Fuc-1-P+ADP

Fuc-1-P+GTP [symbol] GDP-Fuc+PPi

The L-fucokinase/L-fucose-1-phosphate guanylyltransferase belongs to EC classes 2.7.1.52 and 2.7.7.30.

It should be clear that also two separate functional polypeptides, one having L-fucokinase activity and the other having L-fucose-1-phosphate guanylyltransferase activity may be suitable for the method of the present invention.

As used herein, the term “pyrophosphatase” refers to a polypeptide having pyrophosphatase activity, i.e. a polypeptide that catalyzes the following reaction:

PPi+H₂O [symbol] 2 Pi

wherein PPi refers to pyrophosphate and Pi to phosphate.

The pyrophosphatase belongs to EC class 3.6.1.1.

As used herein, the term “glucokinase” refers to a polypeptide having kinase activity, i.e. a kinase that catalyzes the following reactions:

Man+ATP [symbol] Man-6-P+ADP

The glucokinase belongs to the EC class 2.7.1.1.

As used herein, the term “phosphomannomutase” refers to a polypeptide having phosphomannomutase activity, i.e. a phosphomannomutase that catalyzes the following reactions:

Man-6-P [symbol] Man-1-P

The phosphomannomutase belongs to the EC class 5.4.2.8.

As used herein, the term “N-acetylhexosamine-1-kinase” refers to polypeptide having kinase activity, i.e. a polypeptide that catalyzes the following reactions:

Man+ATP [symbol] Man-1-P+ADP

The N-acetylhexosamine-1-kinase belongs to the EC class 2.7.1.162.

As used herein, the term “mannose-1-phosphate guanylyltransferase” refers to a polypeptide having a D-mannose-1-phosphate guanylyltransferase activity, i.e. a polypeptide that catalyzes the following reactions:

Man-1-P+GTP [symbol] GDP-Man+PPi

The mannose-1-phosphate guanylyltransferase belongs to the EC class 2.7.7.13.

As used herein, the term “GDP-mannose 4,6-dehydratase” refers to a polypeptide having GDP-mannose 4,6-dehydratase activity, i.e. a polypeptide that catalyzes the following reactions:

GDP-Man [symbol] GDP-4-dehydro-6-deoxy-alpha-D-mannose

The GDP-mannose 4,6-dehydratase belongs to the EC class 4.2.1.47.

As used herein, the term “GDP-L-fucose synthase” refers to a polypeptide having GDP-L-fucose synthase activity, i.e. a polypeptide that catalyzes the following reactions:

GDP-4-dehydro-6-deoxy-alpha-D-mannose+NADPH [symbol] GDP-Fuc+NADP+

The GDP-L-fucose synthase belongs to the EC class 1.1.1.271 and has the following synonyms and abbreviations GDP-4-keto-6-deoxymannose-3,5-epimerase-4-reductase (WCAG), GDP-4-keto-6-deoxy-D-mannose epimerase-reductase, GDP-4-keto-6-deoxy-D-mannose epimerase/reductase, GDPFuc synthase, GER, GFS, GMER, guanosine diphosphofucose synthetase.

As used herein, the term “co-solvent” refers to an organic compound or a mixture of organic compounds, particularly an organic solvent or a mixture of different solvents, that increases or enhances the solubility of guanosine in water. The person skilled in the art may readily envision that a suitable co-solvent is a solvent in which guanosine has a high solubility, including methanol, ethanol, isopropanol, n-propanol, isobutanol, n-butanol, tert-butanol, acetonitrile, acetone or dimethyl sulfoxide. Preferably the co-solvent is a polar aprotic solvent, such as dimethyl sulfoxide or dimethylformamide. Particularly preferred the co-solvent is dimethyl sulfoxide.

As used herein, “saccharide” refers to but not restricted to monosaccharide, disaccharide, trisaccharide, tetrasaccharide, pentasaccharide, hexasaccharide, heptasaccharide, octasaccharide . . . , oligosaccharide, glycan and polysaccharide. The saccharide comprises preferably monosaccharide units selected from: D-Arabinose, D-Lyxose, D-Ribose, D-Xylose, L-Arabinose, L-Lyxose, L-Ribose, L-Xylose, D-Ribulose, D-Xylulose, L-Ribulose, L-Xylulose, D-Deoxyribose, L-Deoxyribose, D-Erythrose, D-Threose, L-glyCero-D-manno-Heptose, D-glycero-D-manno-Heptose, D-Allose, D-Altrose, D-Glucose, D-Mannose, D-Gulose, D-Idose, D-Galactose, D-Talose, D-psicose, D-fructose, D-sorbose, D-tagatose, 6-Deoxy-L-altrose, 6-Deoxy-D-talose, D-Fucose, L-Fucose, D-Rhamnose, L-Rhamnose, D-Quinovose, Olivose, Tyvelose, Ascarylose, Abequose, Paratose, Digitoxose, Colitose, D-Glucosamine, D-Galactosamine, D-Mannosamine, D-Allosamine, 1-Altrosamine, D-Gulosamine, L-Idosamine, D-Talosamine, N-Acetyl-d-glucosamine, N-Acetyl-D-galactosamine, N-Acetyl-D-mannosamine, N-Acetyl-D-allosamine, N-Acetyl-L-altrosamine, N-Acetyl-D-gulosamine, N-Acetyl-L-idosamine, N-Acetyl-D-talosamine, N-Acetyl-D-fucosamine, N-Acetyl-L-fucosamine, N-Acetyl-L-rhamnosamine, N-Acetyl-D-quinovosamine, D-Glucuronic acid, D-Galacturonic acid, D-Mannuronic acid, D-Alluronic acid, L-Altruronic acid, D-Guluronic acid, L-Guluronic acid, L-Iduronic acid, D-Taluronic acid, Neuraminic acid, N-Acetylneuraminic acid, N-Glycolylneuraminic acid, Apiose, Bacillosamine, Thevetose, Acofriose, Cymarose, Muramic acid, N-Acetylmuramic acid, N-Glycolylmuramic acid, 3-Deoxy-lyxo-heptulosaric acid, Ketodeoxyoctonic acid, and Ketodeoxynononic acid. Preferably the monosaccharide units belong to the following group of α- and β-D/L-carbohydrates comprising or consisting of:

α-D-ribopyranose, α-D-arabinopyranose, α-D-xylopyranose, α-D-lyxopyranose, α-D-allopyranose, α-D-altropyranose, α-D-glucopyranose, α-D-mannpyranose, α-D-glucopyranose, α-D-idopyranose, α-D-galactopyranose, α-D-talopyranose, α-D-psicopyranose, α-D-fructopyranose, α-D-sorbopyranose, α-D-tagatopyranose, α-D-ribofuranose, α-D-arabinofuranose, α-D-xylofuranose, α-D-lyxofuranose, α-D-Allofuranose, α-D-Altrofuranose, α-D-Glucofuranose, α-D-Mannofuranose, α-D-gulofuranose, α-D-idofuranose, α-D-galactofuranose, α-D-talofuranose, α-D-psicofuranose, α-D-fructofuranose, α-D-sorbofuranose, α-D-tagatofuranose, α-D-xylulofuranose, α-D-ribulofuranose, α-D-threofuranose, α-D-rhamnopyranose, α-D-erythrofuranose, α-D-glucosamine, α-D-glucopyranuronic acid, β-D-ribopyranose, β-D-arabinopyranose, β-D-xylopyranose, β-D-lyxopyranose, β-D-allopyranose, β-D-altropyranose, β-D-glucopyranose, β-D-mannpyranose, β-D-glucopyranose, β-D-idopyranose, β-D-galactopyranose, β-D-talopyranose, β-D-psicopyranose, β-D-fructopyranose, β-D-sorbopyranose, β-D-tagatopyranose, β-D-ribofuranose, β-D-arabinofuranose, β-D-xylofuranose, β-D-lyxofuranose, β-D-rhamnopyranose, β-D-allofuranose, β-D-altrofuranose, β-D-glucofuranose, β-D-mannofuranose, β-D-gulofuranose, β-D-idofuranose, β-D-galactofuranose, β-D-talofuranose, β-D-psicofuranose, β-D-fructofuranose, β-D-sorbofuranose, β-D-tagatofuranose, β-D-xylulofuranose, β-D-ribulofuranose, β-D-threofuranose, β-D-erythrofuranose, β-D-glucosamine, β-D-glucopyranuronic acid, α-L-ribopyranose, α-L-arabinopyranose, α-L-xylopyranose, α-L-lyxopyranose, α-L-allopyranose, α-L-altropyranose, α-L-glucopyranose, α-L-mannpyranose, α-L-glucopyranose, α-L-idopyranose, α-L-galactopyranose, α-L-talopyranose, α-L-psicopyranose, α-L-fructopyranose, α-L-sorbopyranose, α-L-tagatopyranose, α-L-rhamnopyranose, α-L-ribofuranose, α-L-arabinofuranose, α-L-xylofuranose, α-L-lyxofuranose, α-L-Allofuranose, α-L-Altrofuranose, α-L-Glucofuranose, α-L-Mannofuranose, α-L-gulofuranose, α-L-idofuranose, α-L-galactofuranose, α-L-talofuranose, α-L-psicofuranose, α-L-fructofuranose, α-L-sorbofuranose, α-L-tagatofuranose, α-L-xylulofuranose, α-L-ribulofuranose, α-L-threofuranose, α-L-erythrofuranose, α-L-glucosamine, α-L-glucopyranuronic acid, β-L-ribopyranose, β-L-arabinopyranose, β-L-xylopyranose, β-L-lyxopyranose, β-L-allopyranose, β-L-altropyranose, β-L-glucopyranose, β-L-mannpyranose, β-L-glucopyranose, β-L-idopyranose, β-L-galactopyranose, β-L-talopyranose, β-L-psicopyranose, β-L-fructopyranose, β-L-sorbopyranose, β-L-tagatopyranose, β-L-ribofuranose, β-L-arabinofuranose, β-L-xylofuranose, β-L-lyxofuranose, β-L-allofuranose, β-L-altrofuranose, β-L-glucofuranose, β-L-mannofuranose, β-L-gulofuranose, β-L-idofuranose, β-L-galactofuranose, β-L-talofuranose, β-L-psicofuranose, β-L-fructofuranose, β-L-sorbofuranose, β-L-tagatofuranose, β-L-xylulofuranose, β-L-ribulofuranose, β-L-threofuranose, β-L-erythrofuranose, β-L-glucosamine, β-L-glucopyranuronic acid, and β-L-rhamnopyranose.

The saccharides are further optionally modified to carry amide, carbonate, carbamate, carbonyl, thiocarbonyl, carboxy, thiocarboxy, ester, thioester, ether, epoxy, hydroxyalkyl, alkylenyl, phenylene, alkenyl, imino, imide, isourea, thiocarbamate, thiourea and/or urea moieties.

As used herein, the term “glycopeptide” refers to a peptide that contains carbohydrate moieties covalently attached to the side chains of the amino acid residues that constitute the peptide. The carbohydrate moieties form side chains and are either O-glycosidic connected to the hydroxy group of a serine or threonine residue or N-glycosidic connected to the amido nitrogen of an asparagine residue.

As used herein, the term “glycoprotein” refers to a polypeptide that contains carbohydrate moieties covalently attached to the side chains of the amino acid residues that constitute the polypeptide. The carbohydrate moieties form side chains and are either O-glycosidic connected to the hydroxy group of a serine or threonine residue or N-glycosidic connected to the amido nitrogen of an asparagine residue.

As used herein, the term “protein” refers to a polypeptide that contains or lacks of carbohydrate moieties covalently attached to the side chains of the amino acid residues that constitute the polypeptide including aglycosylated proteins and glycosylated proteins.

As used herein, the term “peptide” refers to a peptide that contains or lacks of carbohydrate moieties covalently attached to the side chains of the amino acid residues that constitute the peptide, including aglycosylated peptides and glycosylated peptides.

Co-Factor Regeneration

The GDP-L-fucose synthase consumes the cofactor NADPH in the formation of GDP-fucose from GDP-4-dehydro-6-deoxy-α-D-mannose. Thus, in the inventive methods described herein, although not explicitly stated, GDP-fucose is produced from guanosine and D-mannose in the presence of the cofactor NADPH.

Since the co-factors NADPH and NADP⁺ are very expensive, it is advantageous if they are regenerated in the system in order to keep them at catalytic amount and develop a cost effective process.

The enzymes that can be used for regeneration of NADPH are:

Phosphite dehydrogenase EC number 1.20.1.1:

phosphite+NADP⁺ [symbol] phosphate+NADPH

Glucose dehydrogenase EC number 1.1.1.47 or 1.1.1.118:

D-glucose+NADP⁺ [symbol] D-glucono-1,5-lactone+NADPH

Glycerol dehydrogenase EC number 1.1.1.72:

glycerol+NADP⁺ [symbol] glyceraldehyde+NADPH

Glucose-6-phosphate-dehydrogenase (G6PDH) EC number 1.1.1.49:

D-glucose-6-phosphate+NADP⁺ [symbol] 6-phospho-D-glucono-1,5-lactone+NADPH+H⁺

Glutamate dehydrogenase (GLDH) EC number 1.4.1.4:

L-glutamate+H₂O+NADP⁺ [symbol] 2-oxoglutarate+NH₃+NADPH+H⁺

Thus in a preferred embodiment the set of enzymes further includes one of the above listed enzymes that can be used for NADPH regeneration, i.e. a phosphite dehydrogenase, a glycerol dehydrogenase, a glucose dehydrogenase, a glucose-6-phosphate dehydrogenase or a glutamate dehydrogenase. More preferably, the set of enzymes further includes an enzyme selected from a glucose dehydrogenase, a glucose-6-phosphate dehydrogenase and a glutamate dehydrogenase.

Thus the present invention is directed to a method for producing guanosine 5′-diphospho-β-L-fucose from guanosine and L-fucose or D-mannose comprising the following steps:
A) providing a solution comprising
   (i) guanosine and L-fucose or guanosine and D-mannose
   (ii) polyphosphate, adenosine triphosphate a co-solvent for solubilizing guanosine and in case of D-mannose NAPDH; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase; and any of a phosphite dehydrogenase, a glucose dehydrogenase, a glucose-6-phosphate-dehydrogenase and glutamate dehydrogenase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate, NAPDH and the co-solvent.

In a preferred embodiment, the method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate, NADPH and a co-solvent for solubilizing guanosine; and
   providing a set of enzymes comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase; and an enzyme selected from a phosphite dehydrogenase, a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate, NADPH and the co-solvent.

In a particularly preferred embodiment, the method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate, NADPH, L-glutamate and a co-solvent for solubilizing guanosine; and
   providing a set of enzymes comprising a guanosine kinase, a polyphosphate kinase, a glutamate dehydrogenase and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate, NADPH, L-glutamate and the co-solvent.

In a particularly preferred embodiment, the method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate, NADPH, D-glucose and a co-solvent for solubilizing guanosine; and
   providing a set of enzymes comprising a guanosine kinase, a polyphosphate kinase, a glucose-6-phosphate dehydrogenase and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate, NADPH, D-glucose and the co-solvent.

The term “solid support” as used herein refers to an insoluble, functionalized, material to which enzymes or other reagents may be attached or immobilized, directly or via a linker bearing an anchoring group, allowing enzymes to be readily separated (by washing, filtration, centrifugation, etc.) from excess reagents, soluble reaction products, by-products, or solvents. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. A solid support can also consist of magnetic particles. For an overview of suitable support materials for enzyme immobilization see Zdarta et al. Catalysts 2018, 8, 92, and Datta et al. Biotech 2013 3:1-9.

The configuration of a solid support can be in the form of beads, monoliths, spheres, particles, a particle bed, a fiber mat, granules, a gel, a membrane, a hollow-fiber membrane, a mixed-matrix membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

Surprisingly, the inventors have found that by solubilizing guanosine in a co-solvent, such as dimethyl sulfoxide, a nearly complete conversion of guanosine to GDP-fucose was achieved after already three hours (Example 2). Also, the co-solvent did not affect the activity of the enzymes used in the preparation of GDP-fucose. As a co-solvent any compound is suitable that increases or enhances the solubility of guanosine in water or buffer.

Preferably, the co-solvent is an organic solvent selected from the group comprising: methanol, ethanol, isopropanol, n-propanol, isobutanol, n-butanol, tert-butanol, acetonitrile, acetone and dimethyl sulfoxide. Preferably the co-solvent is a polar aprotic solvent such as dimethyl sulfoxide or dimethylformamide. More preferably, the co-solvent is dimethyl sulfoxide. Thus, the present invention is directed to a method for producing guanosine 5′-diphospho-β-L-fucose from guanosine and L-fucose or guanosine and D-mannose comprising the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the co-solvent for solubilizing guanosine is selected from the group comprising methanol, ethanol, isopropanol, n-propanol, isobutanol, n-butanol, tert-butanol, acetonitrile, acetone and dimethyl sulfoxide.

Preferably, the co-solvent is an organic solvent selected from the group comprising: methanol, ethanol, isopropanol, n-propanol, isobutanol, n-butanol, tert-butanol, acetonitrile, acetone and dimethyl sulfoxide. More preferably, the co-solvent is dimethyl sulfoxide. Thus, the present invention is directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein the co-solvent for solubilizing guanosine is selected from the group comprising: methanol, ethanol, isopropanol, n-propanol, isobutanol, n-butanol, tert-butanol, acetonitrile, acetone and dimethyl sulfoxide.

Preferably, the amount of co-solvent for solubilizing guanosine is kept as a low as possible to enable the solubilization of guanosine. Thus, preferably the amount of co-solvent is sufficient to solubilize guanosine completely in the solution provided in step A) of the inventive methods described herein. Alternatively, the amount of co-solvent is sufficient to solubilize at least half of the amount of guanosine in the solution provided in step A) of the inventive methods described herein.

Further, the amount of co-solvent is between 0.01 vol % to 30 vol % based on total volume of the solution provided in step A). More preferably, the amount of co-solvent is between 0.05 vol % to 25 vol % based on total volume of the solution provided in step A). Even more preferably, the amount of co-solvent is between 0.1 vol % to 15 vol % based on total volume of the solution provided in step A). More preferably, the amount of co-solvent is between 0.1 vol % to 10 vol % based on total volume of the solution provided in step A). Most preferably, the amount of co-solvent is between 1 vol % to 5 vol % based on total volume of the solution provided in step A).

Thus, the present invention is directed to a method for producing guanosine 5′-diphospho-β-L-fucose from guanosine and L-fucose or guanosine and D-mannose comprising the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the amount of co-solvent for solubilizing guanosine is between 1 vol % to 5 vol % based on total volume of the solution provided in step A).

Thus, the present invention is directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein the amount of co-solvent for solubilizing guanosine is between 1 vol % to 5 vol % based on total volume of the solution provided in step A).

In one embodiment the method for producing guanosine 5′-diphospho-β-L-fucose from guanosine and L-fucose or guanosine and D-mannose comprising the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the co-solvent is dimethyl sulfoxide and the amount of co-solvent is between 1 vol % to 5 vol % based on total volume of the solution provided in step A).

In one embodiment, the method for producing guanosine 5′-diphospho-β-L-fucose from guanosine and L-fucose comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein the co-solvent is dimethyl sulfoxide and the amount of co-solvent is between 1 vol % to 5 vol % based on total volume of the solution provided in step A).

With the increased solubility, higher concentrated reaction mixtures can be realized, thereby reducing process costs. Thus, the concentration of guanosine and L-fucose in the solution provided in step A) is preferably in the range of 0.01 mM to 100,000 mM. More preferably, the concentration of guanosine and L-fucose is preferably in the range of 0.05 mM to 50,000 mM. More preferably, the concentration of guanosine and L-fucose is preferably in the range of 0.1 mM to 10,000 mM. More preferably, the concentration of guanosine and L-fucose is preferably in the range of 0.2 mM to 5,000 mM.

Thus, the present invention is directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein the concentration of guanosine and L-fucose in the solution provided in step A) is in the range of 0.2 mM to 5,000 mM.

With the increased solubility, higher concentrated reaction mixtures can be realized, thereby reducing process costs. Thus, the concentration of guanosine and D-mannose in the solution provided in step A) is preferably in the range of 0.01 mM to 100,000 mM. More preferably, the concentration of guanosine and D-mannose is preferably in the range of 0.05 mM to 50,000 mM. More preferably, the concentration of guanosine and D-mannose is preferably in the range of 0.1 mM to 10,000 mM. More preferably, the concentration of guanosine and D-mannose is preferably in the range of 0.2 mM to 5,000 mM.

Thus, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,

wherein the concentration of guanosine and D-mannose in the solution provided in step A) is in the range of 0.2 mM to 5,000 mM.

Preferably, the concentration of the enzymes in the set of enzymes is between 0.0001 mg/mL and 100 mg/mL based on the total volume of the solution provided in step A).

As a side product in the reaction of fucose-1-phosphate with guanosine triphosphate to GDP-fucose as well as in the in the reaction of mannose-1-phosphate with guanosine triphosphate to GDP-mannose, pyrophosphate (PPi) is formed. Although pyrophosphate is unstable in aqueous solution, it only slowly hydrolyzes into inorganic phosphate (Pi). A high concentration of pyrophosphate may also lower the activity of the L-fucokinase/L-fucose-1-phosphate guanylyltransferase and mannose-1-phosphate guanylyltransferase enzyme involved in the GDP-fucose formation. The enzyme pyrophosphatase is able to catalyze the hydrolysis of pyrophosphate to phosphate, thereby effectively rendering the GDP-fucose/GDP-mannose formation irreversible. Thus, in a preferred embodiment of the present invention the set of enzymes further comprises a pyrophosphatase.

The method for producing guanosine 5′-diphospho-β-L-fucose from guanosine and L-fucose or guanosine and D-mannose comprising the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase and a pyrophosphatase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexos-amine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent.

The present invention is directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase, and a pyrophosphatase;
B) producing guanosine 5′-diphospho-β-L-fucose from guanosine and fucose in the presence of the set of enzymes, polyphosphate, adenosine triphosphate and the co-solvent.

Reworded, the inventive method for producing guanosine 5′-diphospho-β-L-fucose from guanosine and L-fucose comprises the following steps:
A) providing a solution comprising
   (i) guanosine and L-fucose;
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase, and a pyrophosphatase;
B) producing guanosine 5′-diphospho-β-L-fucose from guanosine and fucose in the presence of the set of enzymes, polyphosphate, adenosine triphosphate and the co-solvent by
   (a) forming fucose-1-phosphate from L-fucose and adenosine triphosphate being catalyzed by a L-fucokinase/L-fucose-1-phosphate guanylyl-transferase,
   (b1) forming guanosine monophosphate from guanosine and adenosine triphosphate being catalyzed by a guanosine kinase;
   (b2′) forming guanosine diphosphate from guanosine monophosphate and polyphosphate being catalyzed by a polyphosphate kinase
   (b2″) forming guanosine triphosphate from guanosine diphosphate and polyphosphate being catalyzed by a polyphosphate kinase,
   (c′) reacting fucose-1-phosphate with guanosine triphosphate to GDP-fucose and pyrophosphate in the presence of a L-fucokinase/L-fucose-1-phosphate guanylyltransferase; and
   (c″) converting pyrophosphate to phosphate in the presence of a pyrophosphatase.

Thus, the present invention is directed to a method for producing guanosine 5′-diphospho-β-L-fucose from guanosine and D-mannose comprising the following steps:
A) providing a solution comprising
   (i) guanosine and D-mannose;
   (ii) polyphosphate, adenosine triphosphate and a co-solvent for solubilizing guanosine; and
   providing a set of enzymes comprising a guanosine kinase, a polyphosphate kinase and either (a) a glucokinase, phosphomannomutase, a mannose-1-phosphate guanylyltransferase, a GDP-mannose-4,6-dehydratase and a GDP-L-fucose-synthase and a pyrophosphatase or (b) an N-acetylhexosamine-1-kinase, a mannose-1-phosphate guanylyltransferase, a GDP-mannose 4,6-dehydratase and a GDP-L-fucose-synthase and a pyrophosphatase;
B) producing guanosine 5′-diphospho-β-L-fucose from guanosine and D-mannose in the presence of the set of enzymes, polyphosphate, adenosine triphosphate and the co-solvent by
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomanno-mutase
(b1) forming guanosine monophosphate (GMP) from guanosine and adenosine triphosphate being catalyzed by a guanosine kinase;
(b2′) forming guanosine diphosphate (GDP) from guanosine monophosphate and polyphosphate being catalyzed by a polyphosphate kinase
(b2″) forming guanosine triphosphate (GTP) from guanosine diphosphate and polyphosphate being catalyzed by a polyphosphate kinase; and
(c′) reacting mannose-1-phosphate with guanosine triphosphate to GDP-mannose and pyrophosphate in the presence of a D-mannose-1-phosphate guanylyl-transferase
(c″) converting pyrophosphate to phosphate in the presence of a pyrophosphatase.
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

Preferably, the inventive method for producing guanosine 5′-diphospho-β-L-fucose from guanosine and L-fucose or guanosine and D-mannose comprising the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase and a pyrophosphatase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexos-amine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the co-solvent is dimethyl sulfoxide.

Preferably, the inventive method for producing guanosine 5′-diphospho-β-L-fucose from guanosine and L-fucose comprises the following steps:
A) providing a solution comprising
   (i) guanosine and L-fucose;
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase, and a pyrophosphatase;
B) producing guanosine 5′-diphospho-β-L-fucose from guanosine and fucose in the presence of the set of enzymes, polyphosphate, adenosine triphosphate and the co-solvent,

wherein the co-solvent is dimethyl sulfoxide.

Preferably, the pyrophosphatase used in the inventive methods described herein is an inorganic pyrophosphatase. Preferably, the pyrophosphatase is an inorganic pyrophosphatase from Pasteurella multocida (PmPpA).

Polyphosphate is able to form stable, water-soluble complexes with metal ions (e.g. Ca²⁺, Mg²⁺, Fe^{2+/3+}) which were initially dissolved in aqueous media. This effect is called sequestration and prevents the bound metal ions from participating in reactions, particularly enzymatic reactions. Therefore, the sequestered metal ions, particularly Mg²⁺ and Mn²⁺, cannot act as co-factor for the enzymes involved in the inventive methods described herein. As the ability of a particular polyphosphate to sequester a particular metal ion decreases with increasing chain length of the polyphosphate, long-chain polyphosphates are preferred in the present invention. More preferred are polyphosphates having at least 14 phosphate residues. Most preferred are polyphosphates having at least 25 phosphate residues.

Thus, the present invention is directed to a method for producing guanosine 5′-diphospho-β-L-fucose from guanosine and L-fucose or guanosine and D-mannose comprising the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the polyphosphate is a long-chain polyphosphate having at least 25 phosphate residues.

Thus, the present invention is directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent;

wherein the polyphosphate is a long-chain polyphosphate having at least 25 phosphate residues Preferably, the method for producing guanosine 5′-diphospho-β-L-fucose from guanosine and L-fucose or guanosine and D-mannose comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the polyphosphate is a long-chain polyphosphate having at least 25 phosphate residues and the co-solvent is dimethyl sulfoxide.

Preferably, the method for producing guanosine 5′-diphospho-β-L-fucose from guanosine and L-fucose comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase, and optionally a pyrophosphatase;
B) producing guanosine 5′-diphospho-β-L-fucose from guanosine and fucose in the presence of the set of enzymes, polyphosphate, adenosine triphosphate and the co-solvent;

wherein the polyphosphate is a long-chain polyphosphate having at least 25 phosphate residues and the co-solvent is dimethyl sulfoxide.

Preferably, the enzymes are present in a single reaction mixture with the other substrates. The mixture may be homogenous (solution) or heterogeneous. The enzymes may be immobilized on a solid support or not. Thus, the guanosine 5′-diphospho-β-L-fucose is produced in a single reaction mixture according to a further aspect of the inventive method.

Thus, the method for producing guanosine 5′-diphospho-β-L-fucose from guanosine and L-fucose or guanosine and D-mannose comprises the following steps:
A) providing a mixture comprising
   (i) guanosine and L-fucose or guanosine and D-mannose
   (ii) polyphosphate, adenosine triphosphate and a co-solvent for solubilizing guanosine; and
   (iii) a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent.

Thus, the method for producing guanosine 5′-diphospho-β-L-fucose from guanosine and L-fucose or guanosine and D-mannose comprises the following steps:
A) providing a mixture comprising
   (i) guanosine and L-fucose or guanosine and D-mannose
   (ii) polyphosphate, adenosine triphosphate, a co-solvent for solubilizing guanosine and in case of D-mannose NADPH; and
   (iii) a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate, NADPH and the co-solvent.

Thus, the method for producing guanosine 5′-diphospho-β-L-fucose from guanosine and L-fucose comprises the following steps:
A) providing a mixture comprising
   (i) guanosine and L-fucose;
   (ii) polyphosphate, adenosine triphosphate and a co-solvent for solubilizing guanosine; and
   (iii) a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent.

Also, the method for producing guanosine 5′-diphospho-β-L-fucose from guanosine and L-fucose comprises the following steps:
A) providing a solution comprising
   (i) guanosine and L-fucose;
   (ii) polyphosphate, adenosine triphosphate and a co-solvent for solubilizing guanosine; and
   (iii) a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent.

Reworded, the method for producing guanosine 5′-diphospho-β-L-fucose from guanosine and L-fucose comprises the following steps:
A) providing a mixture comprising
   (i) guanosine and L-fucose;
   (ii) polyphosphate, adenosine triphosphate and a co-solvent for solubilizing guanosine; and
   (iii) at least three enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the at least three enzymes, polyphosphate, adenosine triphosphate and the co-solvent.

Preferably, the method for producing guanosine 5′-diphospho-β-L-fucose from guanosine and L-fucose or guanosine and D-mannose comprises the following steps:
A) providing a solution comprising
   (i) guanosine and L-fucose or guanosine and D-mannose
   (ii) polyphosphate, adenosine triphosphate and a co-solvent for solubilizing guanosine; and
   (iii) a set of enzymes comprising a guanosine kinase, a polyphosphate kinase, optionally a pyrophosphatase, and either (a) a glucokinase, a phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,

wherein the co-solvent is dimethyl sulfoxide.

Preferably, the method for producing guanosine 5′-diphospho-β-L-fucose from guanosine and L-fucose comprises the following steps:
A) providing a solution comprising
   (i) guanosine and L-fucose;
   (ii) polyphosphate, adenosine triphosphate and a co-solvent for solubilizing guanosine; and
   (iii) a set of enzymes comprising a guanosine kinase, a polyphosphate kinase, a L-fucokinase/L-fucose-1-phosphate guanylyltransferase, and optionally a pyrophosphatase;
B) producing guanosine 5′-diphospho-β-L-fucose from guanosine and fucose in the presence of the set of enzymes, polyphosphate, adenosine triphosphate and the co-solvent;

wherein the co-solvent is dimethyl sulfoxide.

The synthesis of GDP-fucose from guanosine and D-mannose requires NADPH. The cascade is particularly economical if expensive NADPH is recycled in the cascade. Additionally, the reaction equilibrium can be driven towards GDP-fucose by continuous recycling of NADPH from NADP+.

Any of the following enzymes can be applied for recycling of NADPH from NADP+: a phosphite dehydrogenase, a glycerol dehydrogenase, a glucose dehydrogenase (GlcDH), a glucose-6-phosphate dehydrogenase (G6PDH) and a glutamate dehydrogenase (GLDH).

Thus, in a preferred embodiment the set of enzymes further includes any of a glucose dehydrogenase, a glucose-6-phosphate dehydrogenase and a glutamate dehydrogenase.

The present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase and either
      (a) a glucokinase, a phosphomannomutase, a mannose-1-phosphate guanylyltransferase, a GDP-mannose-4,6-dehydratase and a GDP-L-fucose-synthase and an enzyme selected from a glucose dehydrogenase, a glucose-6-phosphate-dehydrogenase and glutamate dehydrogenase; or
      (b) an N-acetylhexosamine-1-kinase, a mannose-1-phosphate guanylyltransferase, a GDP-mannose 4,6-dehydratase and a GDP-L-fucose-synthase, and an enzyme selected from a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably the co-solvent is dimethyl sulfoxide.

Thus, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate, NADPH and a co-solvent for solubilizing guanosine; and
   providing a set of enzymes comprising a guanosine kinase, a polyphosphate kinase and either
      (a) a glucokinase, a phosphomannomutase, a mannose-1-phosphate guanylyltransferase, a GDP-mannose-4,6-dehydratase and a GDP-L-fucose-synthase and any of a glucose dehydrogenase, a glucose-6-phosphate-dehydrogenase and glutamate dehydrogenase; or
      (b) an N-acetylhexosamine-1-kinase, a mannose-1-phosphate guanylyltransferase, a GDP-mannose 4,6-dehydratase and a GDP-L-fucose-synthase, and any of a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably dimethyl sulfoxide by
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomanno-mutase;
(b1) forming guanosine monophosphate (GMP) from guanosine and adenosine triphosphate being catalyzed by a guanosine kinase;
(b2′) forming guanosine diphosphate (GDP) from guanosine monophosphate and polyphosphate being catalyzed by a polyphosphate kinase;
(b2″) forming guanosine triphosphate (GTP) from guanosine diphosphate and polyphosphate being catalyzed by a polyphosphate kinase;
(c′) reacting mannose-1-phosphate with guanosine triphosphate to GDP-mannose and pyrophosphate in the presence of a D-mannose-1-phosphate guanylyl-transferase
(d) forming GDP-4-dehydro-6-deoxy-alpha-D-mannose from GDP-mannose being catalyzed by GDP-mannose-4,6-dehydratase;
(e) forming GDP-fucose from GDP-4-dehydro-6-deoxy-alpha-D-mannose and NADPH being catalyzed by GDP-L-fucose synthase; and
(f) regenerating NADPH from NADP+ being catalyzed by any of a glucose dehydrogenase, a glucose-6-phosphate-dehydrogenase and glutamate dehydrogenase.

Preferred, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase and either
      (a) a glucokinase, a phosphomannomutase, a mannose-1-phosphate guanylyltransferase, a GDP-mannose-4,6-dehydratase and a GDP-L-fucose-synthase, a pyrophosphatase and any of a glucose dehydrogenase, a glucose-6-phosphate-dehydrogenase and glutamate dehydrogenase; or
      (b) an N-acetylhexosamine-1-kinase, a mannose-1-phosphate guanylyltransferase, a GDP-mannose 4,6-dehydratase and a GDP-L-fucose-synthase, a pyrophosphatase, and any of a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably the co-solvent is dimethyl sulfoxide.

Thus, the present invention is directed to a method for producing guanosine 5′-diphospho-β-L-fucose from guanosine and D-mannose comprising the following steps:
A) providing a solution comprising
   (i) guanosine and D-mannose;
   (ii) polyphosphate, adenosine triphosphate, NADPH and a co-solvent for solubilizing guanosine; and
      providing a set of enzymes comprising a guanosine kinase, a polyphosphate kinase and either
      (a) a glucokinase, a phosphomannomutase, a mannose-1-phosphate guanylyltransferase, a GDP-mannose-4,6-dehydratase and a GDP-L-fucose-synthase, a pyrophosphatase and any of a glucose dehydrogenase, a glucose-6-phosphate-dehydrogenase and glutamate dehydrogenase; or
      (b) an N-acetylhexosamine-1-kinase, a mannose-1-phosphate guanylyltransferase, a GDP-mannose 4,6-dehydratase and a GDP-L-fucose-synthase, a pyrophosphatase, and any of a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably dimethyl sulfoxide by
   (a) forming mannose-1-phosphate (Man-1-P) from D-mannose and adenosine triphosphate being catalyzed by a N-acetylhexosamine-1-kinase
      or
      forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase;
   (b1) forming guanosine monophosphate (GMP) from guanosine and adenosine triphosphate being catalyzed by a guanosine kinase;
   (b2′) forming guanosine diphosphate (GDP) from guanosine monophosphate and polyphosphate being catalyzed by a polyphosphate kinase,
   (b2″) forming guanosine triphosphate (GTP) from guanosine diphosphate and polyphosphate being catalyzed by a polyphosphate kinase; and
   (c′) reacting mannose-1-phosphate with guanosine triphosphate to GDP-mannose and pyrophosphate in the presence of a D-mannose-1-phosphate guanylyl-transferase
   (c″) converting pyrophosphate to phosphate in the presence of a pyrophosphatase.
   (d) forming GDP-4-dehydro-6-deoxy-alpha-D-mannose from GDP-mannose being catalyzed by GDP-mannose-4,6-dehydratase; and
   (e) forming GDP-fucose from GDP-4-dehydro-6-deoxy-alpha-D-mannose and NADPH being catalyzed by GDP-L-fucose synthase; and
   (f) regenerating NADPH from NADP+ being catalyzed by any of a glucose dehydrogenase, a glucose-6-phosphate-dehydrogenase and glutamate dehydrogenase.

In the synthesis of GDP-fucose from guanosine and D-mannose, GDP may be in situ produced from GMP by a guanylate kinase (GMK).

Thus, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase; or
      (b) an N-acetylhexosamine-1-kinase, a mannose-1-phosphate guanylyl-transferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent, preferably the co-solvent is dimethyl sulfoxide.

Thus, the present invention is directed to a method for producing guanosine 5′-diphospho-β-L-fucose from guanosine and D-mannose comprising the following steps:
A) providing a solution comprising
   (i) guanosine and D-mannose;
   (ii) polyphosphate, adenosine triphosphate, NADPH and a co-solvent for solubilizing guanosine; and
      providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase; or
      (b) an N-acetylhexosamine-1-kinase, a mannose-1-phosphate guanylyl-transferase, a GDP-mannose 4,6-dehydratase and a GDP-L-fucose-synthase, a pyrophosphatase;
B) producing guanosine 5′-diphospho-β-L-fucose from guanosine and D-mannose in the presence of the set of enzymes, polyphosphate, adenosine triphosphate, NADPH and the co-solvent, preferably dimethyl sulfoxide by
(a) forming mannose-1-phosphate (Man-1-P) from D-mannose and adenosine triphosphate being catalyzed by a N-acetylhexosamine-1-kinase
   or
   forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomanno-mutase;
(b1) forming guanosine monophosphate (GMP) from guanosine and adenosine triphosphate being catalyzed by a guanosine kinase;
(b2′) forming guanosine diphosphate (GDP) from guanosine monophosphate and polyphosphate being catalyzed by a guanylate kinase (GMK),
(b2″) forming guanosine triphosphate (GTP) from guanosine diphosphate and polyphosphate being catalyzed by a polyphosphate kinase; and
(c′) reacting mannose-1-phosphate with guanosine triphosphate to GDP-mannose and pyrophosphate in the presence of a D-mannose-1-phosphate guanylyl-transferase
(d) forming GDP-4-dehydro-6-deoxy-alpha-D-mannose from GDP-mannose being catalyzed by GDP-mannose-4,6-dehydratase; and
(e) forming GDP-fucose from GDP-4-dehydro-6-deoxy-alpha-D-mannose and NADPH being catalyzed by GDP-L-fucose synthase.

Preferred, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate, NADPH and a co-solvent for solubilizing guanosine; and
   providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase, a pyrophosphatase; or
      (b) an N-acetylhexosamine-1-kinase, a mannose-1-phosphate guanylyl-transferase, a GDP-mannose 4,6-dehydratase and a GDP-L-fucose-synthase, a pyrophosphatase;
B) producing guanosine 5′-diphospho-β-L-fucose from guanosine and D-mannose in the presence of the set of enzymes, polyphosphate, adenosine triphosphate and the co-solvent, preferably the co-solvent is dimethyl sulfoxide.

Thus, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate, NADPH and a co-solvent for solubilizing guanosine; and
   providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase, a pyrophosphatase; or
      (b) an N-acetylhexosamine-1-kinase, a mannose-1-phosphate guanylyl-transferase, a GDP-mannose 4,6-dehydratase and a GDP-L-fucose-synthase, a pyrophosphatase;
B) producing guanosine 5′-diphospho-β-L-fucose from guanosine and D-mannose in the presence of the set of enzymes, polyphosphate, adenosine triphosphate, NADPH and the co-solvent, preferably dimethyl sulfoxide by
   (a) forming mannose-1-phosphate (Man-1-P) from D-mannose and adenosine triphosphate being catalyzed by a N-acetylhexosamine-1-kinase
      or
      forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase;
   (b1) forming guanosine monophosphate (GMP) from guanosine and adenosine triphosphate being catalyzed by a guanosine kinase;
   (b2′) forming guanosine diphosphate (GDP) from guanosine monophosphate and polyphosphate being catalyzed by a guanylate kinase (GMK),
   (b2″) forming guanosine triphosphate (GTP) from guanosine diphosphate and polyphosphate being catalyzed by a polyphosphate kinase; and
   (c′) reacting mannose-1-phosphate with guanosine triphosphate to GDP-mannose and pyrophosphate in the presence of a D-mannose-1-phosphate guanylyl-transferase
   (c″) converting pyrophosphate to phosphate in the presence of a pyrophosphatase.
   (d) forming GDP-4-dehydro-6-deoxy-alpha-D-mannose from GDP-mannose being catalyzed by GDP-mannose-4,6-dehydratase; and
   (e) forming GDP-fucose from GDP-4-dehydro-6-deoxy-alpha-D-mannose and NADPH being catalyzed by GDP-L-fucose synthase.

Preferred, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase, and any of a glucose dehydrogenase, a glucose-6-phosphate-dehydrogenase and glutamate dehydrogenase; or
      (b) an N-acetylhexosamine-1-kinase, a mannose-1-phosphate guanylyl-transferase, a GDP-mannose 4,6-dehydratase and a GDP-L-fucose-synthase, and any of a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably the co-solvent is dimethyl sulfoxide.

Thus, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate, NADPH and a co-solvent for solubilizing guanosine; and
   providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase, and any of a glucose dehydrogenase, a glucose-6-phosphate-dehydrogenase and glutamate dehydrogenase; or
      (b) an N-acetylhexosamine-1-kinase, a mannose-1-phosphate guanylyl-transferase, a GDP-mannose 4,6-dehydratase and a GDP-L-fucose-synthase, and any of a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably dimethyl sulfoxide, by
   (a) forming mannose-1-phosphate (Man-1-P) from D-mannose and adenosine triphosphate being catalyzed by a N-acetylhexosamine-1-kinase
      or
      forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase;
   (b1) forming guanosine monophosphate (GMP) from guanosine and adenosine triphosphate being catalyzed by a guanosine kinase;
   (b2′) forming guanosine diphosphate (GDP) from guanosine monophosphate and polyphosphate being catalyzed by a guanylate kinase (GMK),
   (b2″) forming guanosine triphosphate (GTP) from guanosine diphosphate and polyphosphate being catalyzed by a polyphosphate kinase; and
   (c′) reacting mannose-1-phosphate with guanosine triphosphate to GDP-mannose and pyrophosphate in the presence of a D-mannose-1-phosphate guanylyl-transferase;
   (d) forming GDP-4-dehydro-6-deoxy-alpha-D-mannose from GDP-mannose being catalyzed by GDP-mannose-4,6-dehydratase;
   (e) forming GDP-fucose from GDP-4-dehydro-6-deoxy-alpha-D-mannose and NADPH being catalyzed by GDP-L-fucose synthase; and
   (f) regenerating NADPH from NADP+ being catalyzed by any of a glucose dehydrogenase, a glucose-6-phosphate-dehydrogenase and glutamate dehydrogenase.

Also preferred, the present invention is directed to a method for producing guanosine 5′-diphospho-β-L-fucose from guanosine and D-mannose comprising the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase, a pyrophosphatase, and any of a glucose dehydrogenase, a glucose-6-phosphate-dehydrogenase and glutamate dehydrogenase; or
      (b) an N-acetylhexosamine-1-kinase, a mannose-1-phosphate guanylyl-transferase, a GDP-mannose 4,6-dehydratase and a GDP-L-fucose-synthase, a pyrophosphatase, and any of a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably the co-solvent is dimethyl sulfoxide.

Thus, the present invention is directed to a method for producing guanosine 5′-diphospho-β-L-fucose from guanosine and D-mannose comprising the following steps:
A) providing a solution comprising
   (i) guanosine and D-mannose;
   (ii) polyphosphate, adenosine triphosphate, NADPH and a co-solvent for solubilizing guanosine; and
      providing a set of enzymes comprising a guanosine kinase, a polyphosphate kinase, a guanylate kinase (GMK) and either
      (a) a glucokinase, a phosphomannomutase, a mannose-1-phosphate guanylyltransferase, a GDP-mannose-4,6-dehydratase and a GDP-L-fucose-synthase, a pyrophosphatase, and any of a glucose dehydrogenase, a glucose-6-phosphate-dehydrogenase and glutamate dehydrogenase; or
      (b) an N-acetylhexosamine-1-kinase, a mannose-1-phosphate guanylyl-transferase, a GDP-mannose 4,6-dehydratase and a GDP-L-fucose-synthase, a pyrophosphatase, and any of a glucose dehydrogenase, a glucose-6-phosphate-dehydrogenase and glutamate dehydrogenase;
B) producing guanosine 5′-diphospho-β-L-fucose from guanosine and D-mannose in the presence of the set of enzymes, polyphosphate, adenosine triphosphate and the co-solvent, preferably dimethyl sulfoxide by
   (a) forming mannose-1-phosphate (Man-1-P) from D-mannose and adenosine triphosphate being catalyzed by a N-acetylhexosamine-1-kinase
      or
      forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase;
   (b1) forming guanosine monophosphate (GMP) from guanosine and adenosine triphosphate being catalyzed by a guanosine kinase;
   (b2′) forming guanosine diphosphate (GDP) from guanosine monophosphate and polyphosphate being catalyzed by a guanylate kinase (GMK),
   (b2″) forming guanosine triphosphate (GTP) from guanosine diphosphate and polyphosphate being catalyzed by a polyphosphate kinase; and
   (c′) reacting mannose-1-phosphate with guanosine triphosphate to GDP-mannose and pyrophosphate in the presence of a D-mannose-1-phosphate guanylyl-transferase
   (c″) converting pyrophosphate to phosphate in the presence of a pyrophosphatase.
   (d) forming GDP-4-dehydro-6-deoxy-alpha-D-mannose from GDP-mannose being catalyzed by GDP-mannose-4,6-dehydratase; and
   (e) forming GDP-fucose from GDP-4-dehydro-6-deoxy-alpha-D-mannose and NADPH being catalyzed by GDP-L-fucose synthase; and
   (f) regenerating NADPH from NADP+ being catalyzed by any of a glucose dehydrogenase, a glucose-6-phosphate-dehydrogenase and glutamate dehydrogenase.

Due to the recycling of the by-product NADP⁺ in the inventive methods for producing GDP-fucose from guanosine and D-mannose described herein, lower amounts of NADPH are required in the solution provided in step A). Thus, in one embodiment, the molar ratio of NADPH to D-mannose is between 0.01 and 0.5, more preferably between 0.02 and 0.5, more preferably between 0.03 and 0.4, more preferably between 0.03 and 0.3 and most preferably, between 0.05 and 0.2. In one embodiment, the molar ratio of NADPH to D-mannose is 0.05. In one embodiment, the molar ratio of NADPH to D-mannose is 0.1. In one embodiment, the molar ratio of NADPH to D-mannose is 0.2. In one embodiment, the molar ratio of NADPH to D-mannose is 0.5.

The inventive method for producing GDP-fucose can also be carried out with a set of immobilized enzymes. The enzymes are then immobilized on a solid support such that they retain their activity, substrate specificity, stereoselectivity and/or other properties. Suitable solid supports are for instance beads, monoliths, spheres, particles, a particle bed, a fiber mat, granules, a gel, a membrane, a hollow-fiber membrane, a mixed-matrix membrane, a surface or other solid phase material. In one embodiment, each enzyme, i.e. the guanosine kinase, the polyphosphate kinase, the L-fucokinase/L-fucose-1-phosphate guanylyltransferase and optionally the pyrophosphatase, is immobilized on a solid support. In a further embodiment, each enzyme, i.e. the guanosine kinase, the polyphosphate kinase, the glucokinase, the phosphomannomutase, the mannose-1-phosphate guanylyltransferase, the GDP-mannose-4,6-dehydratase the GDP-L-fucose-synthase, optionally the pyrophosphatase, optionally the guanylate kinase, and optionally an enzyme selected from a glucose dehydrogenase, a glucose-6-phosphate dehydrogenase and a glutamate dehydrogenase, is immobilized on a solid support. In a further embodiment, each enzyme, i.e. the guanosine kinase, the polyphosphate kinase, the N-acetylhexosamine-1-kinase, the mannose-1-phosphate guanylyltransferase, the GDP-mannose 4,6-dehydratase the GDP-L-fucose-synthase, optionally the pyrophosphatase, optionally the guanylate kinase, and an enzyme selected from a glucose dehydrogenase, a glucose-6-phosphate dehydrogenase and a glutamate dehydrogenase, is immobilized on a solid support.

In one embodiment, only some of the enzymes of the set of enzymes are immobilized on a solid support. In a further embodiment only one enzyme selected from the set of enzymes comprising a guanosine kinase, a polyphosphate kinase or a combination of polyphosphate kinases e.g. combination 1 D and 2D-ppk2 and ppk3, a L-fucokinase/L-fucose-1-phosphate guanylyltransferase and optionally a pyrophosphatase is immobilized on a solid support. In yet another embodiment, at least one enzyme selected from the set of enzymes comprising a guanosine kinase, a polyphosphate kinase, a L-fucokinase/L-fucose-1-phosphate guanylyltransferase and optionally a pyrophosphatase is immobilized on a solid support. Preferably, the polyphosphate kinase is immobilized on a solid support. Preferably, the guanosine kinase is immobilized on a solid support. Preferably, the L-fucokinase/L-fucose-1-phosphate guanylyltransferase is immobilized on a solid support. Preferably, the pyrophosphatase is immobilized on a solid support.

Thus, the present invention is also directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the set of enzymes is bound or immobilized on a solid support.

Thus, the present invention is also directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein the set of enzymes is bound or immobilized on a solid support.

Thus, the present invention is also directed to a method for producing guanosine 5′-diphospho-β-L-fucose from guanosine and D-mannose comprising the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate, NADPH and the co-solvent,

wherein the set of enzymes is bound or immobilized on a solid support.

Also, the present invention is directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate, and a co-solvent for solubilizing guanosine; and
   providing a set of enzymes comprising a guanosine kinase, a polyphosphate kinase, a pyrophosphatase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the set of enzymes is bound or immobilized on a solid support.

Also, the present invention is directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase and a pyrophosphatase;
B) producing guanosine 5′-diphospho-β-L-fucose from guanosine and fucose in the presence of the set of enzymes, polyphosphate, adenosine triphosphate and the co-solvent,

wherein the set of enzymes is immobilized on a solid support.

Also, the present invention is directed to a method for producing guanosine 5′-diphospho-β-L-fucose from guanosine and D-mannose comprising the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase, a pyrophosphatase and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,

wherein the set of enzymes is immobilized on a solid support.

The present invention is further directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein at least one enzyme of the set of enzymes is immobilized on a solid support.

Thus, the present invention is also directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein at least one enzyme of the set of enzymes is immobilized on a solid support.

Thus, the present invention is also directed to a method for producing guanosine 5′-diphospho-β-L-fucose from guanosine and D-mannose comprising the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase and a glucokinase, phosphomannomutase, a mannose-1-phosphate guanylyltransferase, a GDP-mannose-4,6-dehydratase and a GDP-L-fucose-synthase or a N-acetylhexosamine-1-kinase, a mannose-1-phosphate guanylyl-transferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,

wherein at least one enzyme of the set of enzymes is immobilized on a solid support.

Preferably the enzymes used in the inventive methods described herein are co-immobilized on a solid support. Immobilization of sequentially acting enzymes within a confined space increases catalytic efficiency of conversion due to dramatic reduction in the diffusion time of the substrate. In addition, the in-situ formation of substrates generates high local concentrations that lead to kinetic enhancements and can equate to substantial cost savings. Co-immobilization is usually achieved by mixing the enzymes prior immobilization on a solid support.

Thus, the present invention is also directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the set of enzymes is co-immobilized on a solid support.

Thus, the present invention is also directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein the set of enzymes is co-immobilized on a solid support.

Thus, the present invention is also directed to a method for producing guanosine 5′-diphospho-β-L-fucose from guanosine and D-mannose comprising the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase and a glucokinase, phosphomannomutase, a mannose-1-phosphate guanylyltransferase, a GDP-mannose-4,6-dehydratase and a GDP-L-fucose-synthase or a N-acetylhexosamine-1-kinase, a mannose-1-phosphate guanylyl-transferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,

wherein the set of enzymes is co-immobilized on a solid support.

The present invention is also directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent.

Thus, the present invention is also directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes co-immobilized on a solid support comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent.

Thus, the present invention is also directed to a method for producing guanosine 5′-diphospho-β-L-fucose from guanosine and D-mannose comprising the following steps:
A) providing a solution comprising
   (i) guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase and a glucokinase, phosphomanno-mutase, a mannose-1-phosphate guanylyltransferase, a GDP-mannose-4,6-dehydratase and a GDP-L-fucose-synthase or a N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent.

The present invention is also directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the co-solvent is dimethyl sulfoxide.

Thus, the present invention is also directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes co-immobilized on a solid support comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent,

wherein the co-solvent is dimethyl sulfoxide.

Thus, the present invention is also directed to a method for producing guanosine 5′-diphospho-β-L-fucose from guanosine and D-mannose comprising the following steps:
A) providing a solution comprising
   (i) guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase and a glucokinase, phosphomanno-mutase, a mannose-1-phosphate guanylyltransferase, a GDP-mannose-4,6-dehydratase and a GDP-L-fucose-synthase or a N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,

wherein the co-solvent is dimethyl sulfoxide.

The present invention is further directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the solid support has the form of beads, monoliths, spheres, particles, a particle bed, a fiber mat, granules, a gel, a membrane, a hollow-fiber membrane, a mixed-matrix membrane or a surface.

In such embodiments, the immobilized enzymes can facilitate the production of guanosine 5′-diphospho-β-L-fucose from guanosine and L-fucose, and after the reaction is complete the immobilized enzymes are easily retained (e.g., by retaining beads on which the enzymes are immobilized) and then reused or recycled in subsequent runs. Such immobilized biocatalytic processes allow for further efficiency and cost reduction. In addition, the inventive method can be conducted in a continuous manner by passing the feed solution of step A) through a reactor containing the set of enzymes immobilized on a solid support.

Thus in one embodiment the method for producing guanosine 5′-diphospho-β-L-fucose from guanosine and L-fucose or from guanosine and D-mannose comprises the following steps:
A) providing a feed solution comprising
   (i) guanosine and L-fucose or guanosine and D-mannose,
   (ii) polyphosphate, adenosine triphosphate and a co-solvent for solubilizing guanosine; and
   providing a set of enzymes immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase, wherein the solid support comprising the set of immobilized enzymes is located in a chemical reactor,
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent by continuously passing the feed solution from step A) through the chemical reactor loaded with the solid support comprising the set of immobilized enzymes.

Also the method for producing guanosine 5′-diphospho-β-L-fucose from guanosine and L-fucose or from guanosine and D-mannose comprises the following steps:
A) providing a feed solution comprising
   (i) guanosine and L-fucose or guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase, wherein the solid support comprising the set of co-immobilized enzymes is located in a chemical reactor,
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent by continuously passing the feed solution from step A) through the chemical reactor loaded with the solid support comprising the set of co-immobilized enzymes,

wherein the co-solvent is dimethyl sulfoxide.

Thus, the method for producing guanosine 5′-diphospho-β-L-fucose from guanosine and L-fucose comprises the following steps:
A) providing a feed solution comprising
   (i) guanosine and L-fucose,
   (ii) polyphosphate, adenosine triphosphate and a co-solvent for solubilizing guanosine; and
   providing a set of enzymes co-immobilized on a solid support comprising a guanosine kinase, a polyphosphate kinase, and a L-fucokinase/L-fucose-1-phosphate guanylyltransferase, wherein the solid support comprising the set of co-immobilized enzymes is located in a chemical reactor,
B) producing guanosine 5′-diphospho-β-L-fucose from guanosine and fucose in the presence of the set of enzymes, polyphosphate, adenosine triphosphate and the co-solvent by continuously passing the feed solution from step A) through the chemical reactor loaded with the solid support comprising the set of co-immobilized enzymes,

wherein the co-solvent is dimethyl sulfoxide.

Thus, the method for producing guanosine 5′-diphospho-β-L-fucose from guanosine and D-mannose comprises the following steps:
A) providing a feed solution comprising
   (i) guanosine and L D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase, wherein the solid support comprising the set of co-immobilized enzymes is located in a chemical reactor,
B) producing guanosine 5′-diphospho-β-L-fucose from guanosine and D-mannose in the presence of the set of enzymes, polyphosphate, adenosine triphosphate and the co-solvent by continuously passing the feed solution from step A) through the chemical reactor loaded with the solid support comprising the set of co-immobilized enzymes,

wherein the co-solvent is dimethyl sulfoxide.

Methods of enzyme immobilization are well-known in the art. The enzymes can be bound non-covalently or covalently, such as adsorption, covalent binding, ionic binding, metal binding, crosslinking or crystallization. Various methods for conjugation and immobilization of enzymes to solid supports (e.g., resins, membranes, beads, glass, etc.) are well known in the art and described in e.g.: Yi et al., Process Biochemistry 2007, 42, 895; Martin et al., Applied Microbiology and Biotechnology 2007, 76, 843; Koszelewski et al., Journal of Molecular Catalysis B: Enzymatic, 2010, 63, 39; Truppo et al., Org. Process Res. Dev., 2011, 15, 1033; Hermanson, G. T., Bioconjugate Techniques, Second Edition, Academic Press (2008); Mateo et al., Biotechnology Progress, 2002, 18, 629; and Bioconjugation Protocols: Strategies and Methods, In Methods in Molecular Biology, C. M. Niemeyer ed., Humana Press (2004).

The enzymes used in the inventive methods described herein can be prepared by recombinant methods from bacteria, such as E. coli. The enzyme-containing solutions obtained from cell lysis, which are usually centrifuged and filtered to remove cell debris, can be directly used for immobilizing the enzymes on a solid support. Thus, no further purification step or isolation step is required and the crude cell lysate can be used for immobilizing the enzymes on a solid support such that they retain their activity, substrate specificity, stereoselectivity and/or other properties.

Thus, the present invention is further directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the set of enzymes is immobilized on a solid support from cell lysate.

Also, the present invention is directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the set of enzymes is immobilized on a solid support from crude cell lysate.

Also, the present invention is directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase, optionally a pyrophosphatase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the set of enzymes is co-immobilized on a solid support from cell lysate.

Further, the present invention is directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes co-immobilized on a solid support comprising a guanosine kinase, a polyphosphate kinase, optionally a pyrophosphatase and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent, wherein the set of enzymes is co-immobilized on a solid support from cell lysate, and the co-solvent is dimethyl sulfoxide.

Further, the present invention is directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes comprising a guanosine kinase, a polyphosphate kinase, optionally a pyrophosphatase and in case of L-fucose a L-fucokinase/L-fucose-1-phosphate guanylyltransferase or in case of D-mannose either (a) a glucokinase, phosphomannomutase, a mannose-1-phosphate guanylyl-transferase, a GDP-mannose-4,6-dehydratase and a GDP-L-fucose-synthase or (b) an N-acetylhexosamine-1-kinase, a mannose-1-phosphate guanylyl-transferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein at least one enzyme of the set of enzymes is co-immobilized on a solid support from cell lysate.

Solid supports useful for immobilizing the enzymes used in the method of the present invention include but are not limited to beads or resins comprising polymethacrylate with epoxide functional groups, polymethacrylate with epoxide functional groups, polymethacrylate with amino epoxide functional groups, polymethacrylate with ethylenediamine functional groups, polyacrylic acid with epoxy functional groups, polyacrylic acid with anionic/amino C6 spacer functional groups, polyacrylic acid with anionic/tertiary amine functional groups, polystyrene with anionic/quaternary amine functional groups, polystyrene with cationic/sulphonic functional groups, polyacrylic acid with carboxylic ester functional groups, polystyrene with phenyl functional groups, polymethacrylate with octadecyl functional groups, polystyrene with styrene/methyl functional groups, magnetic silica particles with Ni-NTA functional group, or magnetic nanoparticles with a core of magnetite and a dextran shell with Ni-NTA functional group. Exemplary solid supports useful for immobilizing the enzymes used in the inventive method include, but are not limited to, sepabeads (Resindion): EC-EP, EP403/M, EC-HFA, EC-EA/M and EC-HA; immobeads (ChiralVision) IB-COV1, IB-COV2, IB-COV3, IB-AN11, IB-AN12, IB-AN13, IB-AN14, IB-CAT1, IB-ADS1, IB-ADS2, IB-ADS3 and IB-ADS4; Eupergit (Röhm GmbH & Co. KG) and magnetic particles (micromod GmbH): Nano-mag, Sicastar-6 and Sicastar-1.5.

Preferably, the solid support is composed of a resin or beads selected from: sepabeads (Resindion): EC-EP, EP403/M, EC-EA/M and EC-HA; immobeads (ChiralVision) IB-COV1, IB-COV2, IB-COV3, IB-AN11, IB-CAT1, IB-ADS1, IB-ADS2, IB-ADS3 and IB-ADS4; Eupergit (Röhm GmbH & Co. KG) and magnetic particles (micromod GmbH): Nano-mag, Sicastar-6 and Sicastar-1.5.

Thus, the present invention is further directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the solid support is composed of a resin or beads selected from: sepabeads: EC-EP, EP403/M, EC-EA/M and EC-HA; immobeads: IB-COV1, IB-COV2, IB-COV3, IB-AN11, IB-CAT1, IB-ADS1, IB-ADS2, IB-ADS3 and IB-ADS4; Eupergit and magnetic particles: Nano-mag, Sicastar-6 and Sicastar-1.5.

Also, the present invention is further directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the the solid support is composed of a resin or beads selected from: sepabeads: EC-EP, EP403/M, EC-EA/M and EC-HA; immobeads: IB-COV1, IB-COV2, IB-COV3, IB-AN11, IB-CAT1, IB-ADS1, IB-ADS2, IB-ADS3 and IB-ADS4; Eupergit and magnetic particles: Nano-mag, Sicastar-6 and Sicastar-1.5; and wherein the co-solvent is dimethyl sulfoxide.

Also, the present invention is further directed to a method for producing guanosine 5′-diphospho-β-L-fucose from guanosine and L-fucose or from guanosine and D-mannose comprising the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes immobilized on a solid support comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent,

wherein the solid support is composed of beads or resins comprising polymethacrylate with epoxide functional groups, polymethacrylate with epoxide functional groups, polymethacrylate with amino epoxide functional groups, polymethacrylate with ethylenediamine functional groups, polyacrylic acid with epoxy functional groups, polyacrylic acid with anionic/amino C6 spacer functional groups, polyacrylic acid with anionic/tertiary amine functional groups, polystyrene with anionic/quaternary amine functional groups, polystyrene with cationic/sulphonic functional groups, polyacrylic acid with carboxylic ester functional groups, polystyrene with phenyl functional groups, polymethacrylate with octadecyl functional groups, polystyrene with styrene/methyl functional groups, magnetic silica particles with Ni-NTA functional group, or magnetic nanoparticles with a core of magnetite and a dextran shell with Ni-NTA functional group.

In a further embodiment of the present invention, the method for producing guanosine 5′-diphospho-β-L-fucose comprises the additional step C):
C) isolating the guanosine 5′-diphospho-β-L-fucose.

In a further embodiment of the present invention, the method for producing guanosine 5′-diphospho-β-L-fucose comprises the additional step C):
C) isolating the guanosine 5′-diphospho-β-L-fucose by ion exchange chromatography.

Thus, the present invention is further directed to a method for producing guanosine 5′-diphospho-β-L-fucose from guanosine and L-fucose or guanosine and D-mannose comprising the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
C) isolating the guanosine 5′-diphospho-β-L-fucose.

Thus, the present invention is further directed to a method for producing guanosine 5′-diphospho-β-L-fucose from guanosine and L-fucose comprising the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent; and
C) isolating the guanosine 5′-diphospho-β-L-fucose.

Preferably, the method for producing guanosine 5′-diphospho-β-L-fucose from guanosine and L-fucose or guanosine and D-mannose comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase; and a pyrophosphatase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
C) isolating the guanosine 5′-diphospho-β-L-fucose.

Preferably, the method for producing guanosine 5′-diphospho-β-L-fucose from guanosine and L-fucose comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase; and a pyrophosphatase;
B) producing guanosine 5′-diphospho-β-L-fucose from guanosine and fucose in the presence of the set of enzymes, polyphosphate, adenosine triphosphate and the co-solvent; and
C) isolating the guanosine 5′-diphospho-β-L-fucose.

Preferably, the method for producing guanosine 5′-diphospho-β-L-fucose from guanosine and L-fucose or from guanosine and D-mannose comprises the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes comprising a guanosine kinase, a polyphosphate kinase, optionally a pyrophosphatase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent; and
C) isolating the guanosine 5′-diphospho-β-L-fucose,

wherein at least one enzyme of the set of enzymes is immobilized on a solid support.

Preferably, the method for producing guanosine 5′-diphospho-β-L-fucose from guanosine and L-fucose or guanosine and D-mannose comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase; and optionally a pyrophosphatase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
C) isolating the guanosine 5′-diphospho-β-L-fucose.

Preferably, the method for producing guanosine 5′-diphospho-β-L-fucose from guanosine and L-fucose comprises the following steps:
A) providing a solution comprising
   (i) guanosine and L-fucose,
   (ii) polyphosphate, adenosine triphosphate and a co-solvent for solubilizing guanosine; and
   providing a set of enzymes immobilized on a solid support comprising a guanosine kinase, a polyphosphate kinase, a L-fucokinase/L-fucose-1-phosphate guanylyltransferase; and optionally a pyrophosphatase;
B) producing guanosine 5′-diphospho-β-L-fucose from guanosine and fucose in the presence of the set of enzymes, polyphosphate, adenosine triphosphate and the co-solvent; and
C) isolating the guanosine 5′-diphospho-β-L-fucose.

Preferably, the method for producing guanosine 5′-diphospho-β-L-fucose from guanosine and L-fucose or from guanosine and D-mannose comprises the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes co-immobilized comprising a guanosine kinase, a polyphosphate kinase, optionally a pyrophosphatase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent or from guanosine and D-mannose in the presence of the set of enzymes, polyphosphate, adenosine triphosphate and the co-solvent; and
C) isolating the guanosine 5′-diphospho-β-L-fucose,

wherein the set of enzymes is co-immobilized on a solid support from cell lysate.

Preferably, the method for producing guanosine 5′-diphospho-β-L-fucose from guanosine and L-fucose or guanosine and D-mannose comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase; and a pyrophosphatase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
C) isolating the guanosine 5′-diphospho-β-L-fucose,

wherein the co-solvent is dimethyl sulfoxide.

Preferably, the amount of co-solvent is from 0.01 vol % to 30 vol % based on total volume of the solution provided in step A). Preferably, the co-solvent is dimethyl sulfoxide and the amount of dimethyl sulfoxide is from 0.01 vol % to 30 vol % based on total volume of the solution provided in step A).

Preferably, the polyphosphate is a long-chain polyphosphate having at least 25 phosphate residues.

Preferably, the concentration of guanosine and L-fucose or guanosine and D-mannose in the solution provided in step A) is in the range of 0.2 mM to 5,000 mM.

Preferably, the method for producing guanosine 5′-diphospho-β-L-fucose from guanosine and L-fucose comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase; and optionally a pyrophosphatase;
B) producing guanosine 5′-diphospho-β-L-fucose from guanosine and fucose in the presence of the set of enzymes, polyphosphate, adenosine triphosphate and the co-solvent; and
C) isolating the guanosine 5′-diphospho-β-L-fucose, wherein the co-solvent is dimethyl sulfoxide.

Preferably, the amount of co-solvent is from 0.01 vol % to 30 vol % based on total volume of the solution provided in step A). Preferably, the co-solvent is dimethyl sulfoxide and the amount of dimethyl sulfoxide is from 0.01 vol % to 30 vol % based on total volume of the solution provided in step A).

Preferably, the polyphosphate is a long-chain polyphosphate having at least 25 phosphate residues.

Preferably, the concentration of guanosine and L-fucose in the solution provided in step A) is in the range of 0.2 mM to 5,000 mM.

Fucosylated Saccharides, Fucosylated Glycopeptides, Fucosylated Glycoproteins, Fucosylated Proteins, Fucosylated Peptides and Fucosylated Small Molecules, Such as Stevia.

In a further aspect of the present invention the inventive methods described herein are useful for producing fucosylated saccharides, fucosylated glycopeptides, fucosylated glycoproteins, fucosylated proteins, fucosylated peptides or fucosylated small molecules, e.g. stevia.

Thus, in one embodiment of the present invention the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent, and
D) producing a fucosylated saccharide, fucosylated glycopeptide, fucosylated glycoprotein, fucosylated protein, fucosylated peptide or fucosylated small molecule from guanosine 5′-diphospho-β-L-fucose and a saccharide, glycopeptide, glycoprotein, protein, peptide or small molecule by forming an
O-glycosidic bond between guanosine 5′-diphospho-β-L-fucose and an available hydroxyl group of the saccharide, glycopeptide, glycoprotein, protein, peptide or small molecule in the presence of a fucosyltransferase.

Thus, in one embodiment of the present invention the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a fucosyltransferase and a L-fucokinase/L-fucose-1-phosphate guanylyl-transferase;
B) producing guanosine 5′-diphospho-β-L-fucose from guanosine and fucose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-L-fucose and a saccharide, glycopeptide, glycoprotein, protein, peptide or small molecule by forming an
O-glycosidic bond between guanosine 5′-diphospho-β-L-fucose and an available hydroxyl group of the saccharide, glycopeptide, glycoprotein, protein, peptide or small molecule in the presence of a fucosyltransferase.

Thus, in one embodiment of the present invention the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and mannose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-L-fucose and a saccharide, glycopeptide, glycoprotein, protein, peptide or small molecule by forming an
O-glycosidic bond between guanosine 5′-diphospho-β-L-fucose and an available hydroxyl group of the saccharide, glycopeptide, glycoprotein, protein, peptide or small molecule in the presence of a fucosyltransferase.

In one embodiment of the present invention the method for producing a fucosylated saccharide, a fucosylated glycopeptide a fucosylated glycoprotein, a fucosylated protein, a fucosylated peptide or a fucosylated small molecule comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyltransferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
C) isolating the guanosine 5′-diphospho-β-L-fucose; and
D) producing a fucosylated saccharide, fucosylated glycopeptide, fucosylated glycoprotein, fucosylated protein, fucosylated peptide or fucosylated small molecule from guanosine 5′-diphospho-β-L-fucose and a saccharide, glycopeptide, glycoprotein, protein, peptide or small molecule by forming an
O-glycosidic bond between guanosine 5′-diphospho-β-L-fucose and an available hydroxyl group of the saccharide, glycopeptide, glycoprotein, protein, peptide or small molecule in the presence of a fucosyltransferase.

In one embodiment of the present invention the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a fucosyltransferase and a L-fucokinase/L-fucose-1-phosphate guanylyltransferase;
B) producing guanosine 5′-diphospho-β-L-fucose from guanosine and fucose in the presence of the set of enzymes, polyphosphate, adenosine triphosphate and the co-solvent;
C) isolating the guanosine 5′-diphospho-β-L-fucose; and
D) producing a fucosylated saccharide, fucosylated glycopeptide, fucosylated glycoprotein, fucosylated protein, fucosylated peptide or fucosylated small molecule from guanosine 5′-diphospho-β-L-fucose and a saccharide, glycopeptide, glycoprotein, protein, peptide or small molecule by forming an
O-glycosidic bond between guanosine 5′-diphospho-β-L-fucose and an available hydroxyl group of the saccharide, glycopeptide, glycoprotein, protein, peptide or small molecule in the presence of a fucosyltransferase.

In one embodiment of the present invention the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and mannose in the presence of the set of enzymes, polyphosphate, adenosine triphosphate and the co-solvent;
C) isolating the guanosine 5′-diphospho-β-L-fucose; and
D) producing a fucosylated saccharide, fucosylated glycopeptide, fucosylated glycoprotein, fucosylated protein, fucosylated peptide or fucosylated small molecule from guanosine 5′-diphospho-β-L-fucose and a saccharide, glycopeptide, glycoprotein, protein, peptide or small molecule by forming an
O-glycosidic bond between guanosine 5′-diphospho-β-L-fucose and an available hydroxyl group of the saccharide, glycopeptide, glycoprotein, protein, peptide or small molecule in the presence of a fucosyltransferase.

The fucosyltransferase catalyzes the reaction of GDP-fucose with an available hydroxyl group of a saccharide, glycopeptide, glycoprotein, protein, peptide or small molecule, thereby forming a fucosylated saccharide, fucosylated glycopeptide, fucosylated glycoprotein, a fucosylated protein, a fucosylated peptide or a fucosylated small molecule and guanosine diphosphate (GDP) as side product. GDP being an intermediate product formed in step B), specifically in step (b2′) can then be reused or recycled.

Thus, in one embodiment of the present invention the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a fucosyltransferase and a L-fucokinase/L-fucose-1-phosphate guanylyl-transferase;
B) producing guanosine 5′-diphospho-β-L-fucose from guanosine and fucose in the presence of the set of enzymes, polyphosphate, adenosine triphosphate and the co-solvent by
   (a) forming fucose-1-phosphate from L-fucose and adenosine triphosphate being catalyzed by a L-fucokinase/L-fucose-1-phosphate guanylyl-transferase,
   (b1) forming guanosine monophosphate from guanosine and adenosine triphosphate being catalyzed by a guanosine kinase;
   (b2′) forming guanosine diphosphate from guanosine monophosphate and polyphosphate being catalyzed by a polyphosphate kinase
   (b2″) forming guanosine triphosphate from guanosine diphosphate and polyphosphate being catalyzed by a polyphosphate kinase; and
   (c) reacting fucose-1-phosphate with guanosine triphosphate to GDP-fucose in the presence of a L-fucokinase/L-fucose-1-phosphate guanylyltransferase;
D) producing a fucosylated saccharide, fucosylated glycopeptide, fucosylated glycoprotein, fucosylated protein, fucosylated peptide or fucosylated small molecule from guanosine 5′-diphospho-β-I-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase; and
E) recycling the in-situ formed guanosine diphosphate to form guanosine triphosphate.

Thus, in one embodiment of the present invention the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and D-mannose;
   (ii) polyphosphate, adenosine triphosphate and a co-solvent for solubilizing guanosine; and
   providing a set of enzymes comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase and a pyrophosphatase or (b) an N-acetylhexosamine-1-kinase, a mannose-1-phosphate guanylyltransferase, a GDP-mannose 4,6-dehydratase and a GDP-L-fucose-synthase,
B) producing guanosine 5′-diphospho-β-L-fucose from guanosine and D-mannose in the presence of the set of enzymes, polyphosphate, adenosine triphosphate and the co-solvent by
   (a) forming mannose-1-phosphate (Man-1-P) from D-mannose and adenosine triphosphate being catalyzed by a N-acetylhexosamine-1-kinase
      or
      forming mannose-6-phosphate (Man-6-P) from D-mannose and adenosine triphosphate being catalyzed by glucokinase and forming mannose-1-phosphat (Man-1-P) from mannose-6-phosphate being catalyzed by phosphomannomutase
   (b1) forming guanosine monophosphate (GMP) from guanosine and adenosine triphosphate being catalyzed by a guanosine kinase;
   (b2′) forming guanosine diphosphate (GDP) from guanosine monophosphate and polyphosphate being catalyzed by a polyphosphate kinase
   (b2″) forming guanosine triphosphate (GTP) from guanosine diphosphate and polyphosphate being catalyzed by a polyphosphate kinase; and
   (c′) reacting mannose-1-phosphate with guanosine triphosphate to GDP-mannose and pyrophosphate in the presence of a D-mannose-1-phosphate guanylyltransferase
   (c″) converting pyrophosphate to phosphate in the presence of a pyrophosphatase.
   (d) forming GDP-4-dehydro-6-deoxy-alpha-D-mannose from GDP-mannose being catalyzed by GDP-mannose-4,6-dehydratase; and
   (e) forming GDP-fucose from GDP-4-dehydro-6-deoxy-alpha-D-mannose and NADPH being catalyzed by GDP-L-fucose synthase.
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase; and
E) recycling the in-situ formed guanosine diphosphate to form guanosine triphosphate.

Due to the recycling of the by-product guanosine diphosphate in the inventive fucosylation methods described herein, lower amounts of guanosine are required in the solution provided in step A). Thus, in one embodiment, the molar ratio of guanosine to L-fucose or guanosine to D-mannose is between 0.01 and 0.5, more preferably between 0.02 and 0.5, more preferably between 0.03 and 0.4, more preferably between 0.03 and 0.3 and most preferably, between 0.05 and 0.2. In one embodiment, the molar ratio of guanosine to L-fucose or guanosine to D-mannose is 0.05. In one embodiment, the molar ratio of guanosine to L-fucose or guanosine to D-mannose is 0.1. In one embodiment, the molar ratio of guanosine to L-fucose or guanosine to D-mannose is 0.2. In one embodiment, the molar ratio of guanosine to L-fucose or guanosine to D-mannose is 0.5.

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phospho-mannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an O-glycosidic bond between guanosine 5′-diphospho-β-l-fucose and an available hydroxyl group of the saccharide, glycopeptide, glycoprotein, protein, peptide or small molecule in the presence of a fucosyltransferase,
F) isolating the fucosylated saccharide, fucosylated glycopeptide, fucosylated glycoprotein, fucosylated protein, fucosylated peptide or fucosylated small molecule.

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a fucosyltransferase and a L-fucokinase/L-fucose-1-phosphate guanylyl-transferase;
B) producing guanosine 5′-diphospho-β-L-fucose from guanosine and fucose in the presence of the set of enzymes, polyphosphate, adenosine triphosphate and the co-solvent;
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase; and
F) isolating the fucosylated saccharide, fucosylated glycopeptide, fucosylated glycoprotein, fucosylated protein, fucosylated peptide or fucosylated small molecule.

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetyl-hexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
C) isolating the guanosine 5′-diphospho-β-L-fucose; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase,
wherein the co-solvent is dimethyl sulfoxide.

Preferably, the amount of co-solvent is from 0.01 vol % to 30 vol % based on total volume of the solution provided in step A). Preferably, the co-solvent is dimethyl sulfoxide and the amount of dimethyl sulfoxide is from 0.01 vol % to 30 vol % based on total volume of the solution provided in step A).

Preferably, the polyphosphate is a long-chain polyphosphate having at least 25 phosphate residues.

Preferably, the concentration of guanosine and L-fucose or guanosine and D-mannose in the solution provided in step A) is in the range of 0.2 mM to 5,000 mM.

Preferably, the concentration of the enzymes in the set of enzymes is between 0.0001 mg/mL and 100 mg/mL based on the total volume of the solution provided in step A).

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a fucosyltransferase and a L-fucokinase/L-fucose-1-phosphate guanylyl-transferase;
B) producing guanosine 5′-diphospho-β-L-fucose from guanosine and fucose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase,

wherein the co-solvent is dimethyl sulfoxide.

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and mannose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase,

wherein the co-solvent is dimethyl sulfoxide.

Preferably, the amount of co-solvent is from 0.01 vol % to 30 vol % based on total volume of the solution provided in step A). Preferably, the co-solvent is dimethyl sulfoxide and the amount of dimethyl sulfoxide is from 0.01 vol % to 30 vol % based on total volume of the solution provided in step A).

Preferably, the polyphosphate is a long-chain polyphosphate having at least 25 phosphate residues.

Preferably, the concentration of guanosine and L-fucose in the solution provided in step A) is in the range of 0.2 mM to 5,000 mM.

Preferably, the concentration of the enzymes in the set of enzymes is between 0.0001 mg/mL and 100 mg/mL based on the total volume of the solution provided in step A).

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase; and a pyrophosphatase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
C) isolating the guanosine 5′-diphospho-β-L-fucose; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase.

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase, a fucosyl-transferase and a pyrophosphatase;
B) producing guanosine 5′-diphospho-β-L-fucose from guanosine and fucose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase.

Preferably, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose or guanosine and D-mannose,
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an O-glycosidic bond between guanosine 5′-diphospho-β-l-fucose and an available hydroxyl group of the saccharide, glycopeptide, glycoprotein, protein, peptide or small molecule in the presence of a fucosyltransferase,

wherein at least one enzyme of the set of enzymes is immobilized on a solid support.

In one embodiment, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a L-fucokinase/L-fucose-1-phosphate guanylyltransferase, and optionally a pyrophosphatase;
B) producing guanosine 5′-diphospho-β-L-fucose from guanosine and fucose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an O-glycosidic bond between guanosine 5′-diphospho-β-l-fucose and an available hydroxyl group of the saccharide, glycopeptide, glycoprotein, protein, peptide or small molecule in the presence of a fucosyltransferase,

wherein at least one enzyme of the set of enzymes is immobilized on a solid support.

In one embodiment, the method for producing a fucosylated saccharide, a fucosylated glycopeptide, a fucosylated glycoprotein, a fucosylated protein, a fucosylated peptide or a fucosylated small molecule comprises the following steps:
A) providing a solution comprising
   (i) guanosine and D-mannose,
   (ii) polyphosphate, adenosine triphosphate and a co-solvent for solubilizing guanosine; and
   providing a set of enzymes comprising a guanosine kinase, a polyphosphate kinase, and a glucokinase, phosphomannomutase, a mannose-1-phosphate guanylyltransferase, a GDP-mannose-4,6-dehydratase and a GDP-L-fucose-synthase or a N-acetylhexosamine-1-kinase, a mannose-1-phosphate guanylyl-transferase, a GDP-mannose 4,6-dehydratase and a GDP-L-fucose-synthase;
B) producing guanosine 5′-diphospho-β-L-fucose from guanosine and fucose in the presence of the set of enzymes, polyphosphate, adenosine triphosphate and the co-solvent; and
D) producing a fucosylated saccharide, fucosylated glycopeptide, fucosylated glycoprotein, fucosylated protein, fucosylated peptide or fucosylated small molecule from guanosine 5′-diphospho-β-l-fucose and a saccharide, glycopeptide, glycoprotein, protein, peptide or small molecule by forming an
O-glycosidic bond between guanosine 5′-diphospho-β-l-fucose and an available hydroxyl group of the saccharide, glycopeptide, glycoprotein, protein, peptide or small molecule in the presence of a fucosyltransferase,

wherein at least one enzyme of the set of enzymes is immobilized on a solid support.

In one embodiment, fucosylated milk saccharides are produced by the inventive methods described herein. Thus, in one embodiment the inventive method comprises the following steps:
A) providing a solution comprising
   (i) guanosine and L-fucose,
   (ii) polyphosphate, adenosine triphosphate and a co-solvent for solubilizing guanosine; and
   providing a set of enzymes comprising a guanosine kinase, a polyphosphate kinase, a fucosyltransferase and a L-fucokinase/L-fucose-1-phosphate guanylyl-transferase;
B) producing guanosine 5′-diphospho-β-L-fucose from guanosine and fucose in the presence of the set of enzymes, polyphosphate, adenosine triphosphate and the co-solvent; and
D) producing a fucosylated milk saccharide from guanosine 5′-diphospho-β-L-fucose and a milk saccharide by forming an O-glycosidic bond between guanosine 5′-diphospho-β-L-fucose and an available hydroxyl group of the milk saccharide, in the presence of a fucosyltransferase.

In one embodiment, fucosylated milk saccharides are produced by the inventive methods described herein. Thus, in one embodiment the inventive method comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing a fucosylated milk saccharide from guanosine 5′-diphospho-β-L-fucose and a milk saccharide by forming an O-glycosidic bond between guanosine 5′-diphospho-β-L-fucose and an available hydroxyl group of the milk saccharide, in the presence of a fucosyltransferase.

Preferably the fucosylated milk saccharides are selected from the group comprising 2′-fucosyllactose, 3-fucosyllactose, lacto-N-fucopentaose I, lacto-N-fucopentaose III, lacto-N-difucohexaose I and lacto-N-difucohexaose II (see FIG. 6).

In a preferred embodiment, 2′-fucosyllactose is produced by the inventive methods described herein (FIG. 10). Thus, in one embodiment the inventive method comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 2′-fucosyllactose from guanosine 5′-diphospho-β-L-fucose and lactose by forming an O-glycosidic bond between guanosine 5′-diphospho-β-L-fucose and an available hydroxyl group of the lactose, in the presence of a fucosyltransferase.

In preferred embodiment, 2′-fucosyllactose is produced by the inventive methods described herein. Thus, in one embodiment the inventive method comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 2′-fucosyllactose from guanosine 5′-diphospho-β-L-fucose and lactose by forming an O-glycosidic bond between guanosine 5′-diphospho-β-L-fucose and an available hydroxyl group of the lactose, in the presence of type 1 galactoside-alpha-(1,2)-fucosyltransferase.

In preferred embodiment, the method for producing 2′-fucosyllactose comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 2′-Fucosyllactose from guanosine 5′-diphospho-β-L-fucose and lactose by forming an O-glycosidic bond between guanosine 5′-diphospho-β-L-fucose and an available hydroxyl group of the lactose, in the presence of type 1 galactoside-alpha-(1,2)-fucosyltransferase; and
E) recycling the in-situ formed guanosine diphosphate to form guanosine triphosphate.

In a preferred embodiment, 3-fucosyllactose is produced by the inventive methods described herein (FIGS. 18 and 19). Thus, in one embodiment the inventive method comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 3-fucosyllactose from guanosine 5′-diphospho-β-L-fucose and lactose by forming an O-glycosidic bond between guanosine 5′-diphospho-β-L-fucose and an available hydroxyl group of the lactose, in the presence of a fucosyltransferase.

In preferred embodiment, 3-fucosyllactose is produced by the inventive methods described herein. Thus, in one embodiment the inventive method comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 3-fucosyllactose from guanosine 5′-diphospho-β-L-fucose and lactose by forming an O-glycosidic bond between guanosine 5′-diphospho-β-L-fucose and an available hydroxyl group of the lactose, in the presence of 3/4-fucosyltransferase.

In preferred embodiment, 3-fucosyllactose is produced by the inventive methods described herein. Thus, in one embodiment the inventive method comprises the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 3-fucosyllactose from guanosine 5′-diphospho-β-L-fucose and lactose by forming an O-glycosidic bond between guanosine 5′-diphospho-β-L-fucose and an available hydroxyl group of the lactose, in the presence of 3/4-fucosyltransferase.

In preferred embodiment, the method for producing 3-fucosyllactose comprises the following steps:
A) providing a solution comprising
   (i) guanosine and L-fucose or guanosine and D-mannose
   (ii) polyphosphate, adenosine triphosphate and a co-solvent for solubilizing guanosine; and
   providing a set of enzymes comprising a guanosine kinase, a polyphosphate kinase, and in case of L-fucose a L-fucokinase/L-fucose-1-phosphate guanylyl-transferase or in case of D-mannose either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and L-fucose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 3-fucosyllactose from guanosine 5′-diphospho-β-L-fucose and lactose by forming an O-glycosidic bond between guanosine 5′-diphospho-β-L-fucose and an available hydroxyl group of the lactose, in the presence of 3/4-fucosyltransferase; and
E) recycling the in-situ formed guanosine diphosphate to form guanosine triphosphate.

Due to the recycling of the by-product guanosine diphosphate in the inventive fucosylation methods described herein, lower amounts of guanosine are required in the solution provided in step A). Thus, in one embodiment, the molar ratio of guanosine to L-fucose or guanosine to D-mannose is between 0.01 and 0.5, more preferably between 0.02 and 0.5, more preferably between 0.03 and 0.4, more preferably between 0.03 and 0.3 and most preferably, between 0.05 and 0.2. In one embodiment, the molar ratio of guanosine to L-fucose or guanosine to D-mannose is 0.05. In one embodiment, the molar ratio of guanosine to L-fucose or guanosine to D-mannose is 0.1. In one embodiment, the molar ratio of guanosine to L-fucose or guanosine to D-mannose is 0.2. In one embodiment, the molar ratio of guanosine to L-fucose or guanosine to D-mannose is 0.5.

In a preferred embodiment the inventive method comprises the following steps:
A) providing a solution comprising
   (i) guanosine and D-mannose
   (ii) polyphosphate, adenosine triphosphate, NADPH, L-glutamate and a co-solvent for solubilizing guanosine; and
   providing a set of enzymes comprising a guanosine kinase, a polyphosphate kinase, a glutamate dehydrogenase, and either (a) a glucokinase, phosphomanno-mutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent or from guanosine and D-mannose in the presence of the set of enzymes, polyphosphate, adenosine triphosphate and the co-solvent,
D) producing 3-fucosyllactose from guanosine 5′-diphospho-β-L-fucose and lactose by forming an O-glycosidic bond between guanosine 5′-diphospho-β-L-fucose and an available hydroxyl group of the lactose, in the presence of 3/4-fucosyltransferase,
E) recycling the in-situ formed guanosine diphosphate to form guanosine triphosphate,
F) regenerating NADPH from in situ formed NADP+ being catalyzed by a glutamate dehydrogenase.

In a preferred embodiment the inventive method comprises the following steps:
A) providing a solution comprising
   (i) guanosine and D-mannose
   (ii) polyphosphate, adenosine triphosphate, NADPH, D-glucose and a co-solvent for solubilizing guanosine; and
   providing a set of enzymes comprising a guanosine kinase, a polyphosphate kinase, a glucose-6-phosphate dehydrogenase, and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate, NADPH, D-glucose and the co-solvent or from guanosine and D-mannose in the presence of the set of enzymes, polyphosphate, adenosine triphosphate and the co-solvent,
D) producing 3-fucosyllactose from guanosine 5′-diphospho-β-L-fucose and lactose by forming an O-glycosidic bond between guanosine 5′-diphospho-β-L-fucose and an available hydroxyl group of the lactose, in the presence of 3/4-fucosyltransferase,
E) recycling the in-situ formed guanosine diphosphate to form guanosine triphosphate,
F) regenerating NADPH from in situ formed NADP+ being catalyzed by a glutamate dehydrogenase.

Due to the recycling of the by-product NADP⁺ in the inventive methods for producing GDP-fucose from guanosine and D-mannose described herein, lower amounts of NADPH are required in the solution provided in step A). Thus, in one embodiment, the molar ratio of NADPH to D-mannose is between 0.01 and 0.5, more preferably between 0.02 and 0.5, more preferably between 0.03 and 0.4, more preferably between 0.03 and 0.3 and most preferably, between 0.05 and 0.2. In one embodiment, the molar ratio of NADPH to D-mannose is 0.05. In one embodiment, the molar ratio of NADPH to D-mannose is 0.1. In one embodiment, the molar ratio of NADPH to D-mannose is 0.2. In one embodiment, the molar ratio of NADPH to D-mannose is 0.5.

Production of L-Fucose

Since L-Fucose can be considered as an expensive sugar (in very large scales) the synthesis of L-fucose from D-mannose is further provided with the method of the present invention. First GDP-fucose is produced from D-mannose and guanosine as described herein. Afterwards, by addition of a fucosyltransferase (EC 2.4.1.344 or EC 2.4.1.69) or a fucosidase (EC 3.2.1.51) without addition of any acceptor—GDP-fucose will be hydrolyzed to fucose, since transferase can work reversibly. Reaction scheme is shown in FIG. 9. Thus, L-fucose can be produced from low cost substrates such as D-mannose and guanosine.

Alternatively, L-fucose is produced simply by heating the guanosine 5′-diphospho-β-L-fucose at the temperature in a range of 80 to 100° C. as described in the Example 10. Preferably the guanosine 5′-diphospho-β-L-fucose is heated at this temperature for 0.5 to 3 hours, preferably 0.5 to 2 hours, more preferably 0.5 to 1.5 hours, most preferably for 1 hour.

Thus, in one embodiment, L-Fucose is produced by the inventive method as described herein starting from guanosine and D-mannose. Thus, in one embodiment the inventive method comprises the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase; and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,
D) producing L-fucose from guanosine 5′-diphospho-β-L-fucose in the presence of a fucosyltransferase and in the absence of an acceptor; or producing L-fucose by heating guanosine 5′-diphospho-β-L-fucose at a temperature in a range of 80 to 100° C.

Thus, in one embodiment, L-Fucose is produced by the inventive method as described herein starting from guanosine and D-mannose. Thus, in one embodiment the inventive method comprises the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase; and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,
D) producing L-fucose from guanosine 5′-diphospho-β-L-fucose in the presence of type 1 galactoside-alpha-(1,2)-fucosyltransferase and in the absence of an acceptor, or
   producing L-fucose by heating guanosine 5′-diphospho-β-L-fucose at a temperature in a range of 80 to 100° C.

Thus, in one embodiment the inventive method comprises the following steps:
A) providing a solution comprising
   (i) guanosine and D-mannose
   (ii) polyphosphate, adenosine triphosphate and a co-solvent for solubilizing guanosine; and
   providing a set of enzymes comprising a guanosine kinase, a polyphosphate kinase; and either (a) a glucokinase, phosphomannomutase, a mannose-1-phosphate guanylyltransferase, a GDP-mannose-4,6-dehydratase and a GDP-L-fucose-synthase or (b) an N-acetylhexosamine-1-kinase, a mannose-1-phosphate guanylyltransferase, a GDP-mannose 4,6-dehydratase and a GDP-L-fucose-synthase;
B) producing guanosine 5′-diphospho-β-L-fucose from guanosine and D-mannose in the presence of the set of enzymes, polyphosphate, adenosine triphosphate and the co-solvent,
D) producing L-fucose from guanosine 5′-diphospho-β-L-fucose in the presence of type 1 galactoside-alpha-(1,2)-fucosyltransferase and in the absence of an acceptor, or
   producing L-fucose by heating guanosine 5′-diphospho-β-L-fucose at a temperature in a range of 80 to 100° C., and
E) recycling the in-situ formed guanosine diphosphate to form guanosine triphosphate.

Due to the recycling of the by-product guanosine diphosphate in the inventive fucosylation methods described herein, lower amounts of guanosine are required in the solution provided in step A). Thus, in one embodiment, the molar ratio of guanosine to L-fucose or guanosine to D-mannose is between 0.01 and 0.5, more preferably between 0.02 and 0.5, more preferably between 0.03 and 0.4, more preferably between 0.03 and 0.3 and most preferably, between 0.05 and 0.2. In one embodiment, the molar ratio of guanosine to L-fucose or guanosine to D-mannose is 0.05. In one embodiment, the molar ratio of guanosine to L-fucose or guanosine to D-mannose is 0.1. In one embodiment, the molar ratio of guanosine to L-fucose or guanosine to D-mannose is 0.2. In one embodiment, the molar ratio of guanosine to L-fucose or guanosine to D-mannose is 0.5.

As used herein, the term “acceptor” refers to any molecule or macromolecule, including a saccharide, peptide or protein that is capable of being fucosylated by a fucosyltransferase, i.e. acting as a substrate in the reaction catalyzed by a fucosyl-transferase as described herein. Thus, the acceptor prevents the hydrolysis of GDP-fucose by fucosyltransferase in aqueous media. Particularly, the acceptor is a glycosyl acceptor which is nucleophilic. Preferably, the glycosyl acceptor has an oxygen-carbon-, nitrogen-, or sulfur-based nucleophilic group which can form a covalent glycosidic bond with the fucosyl moiety of GDP-fucose.

In any of the above-described inventive methods, preferably the glucokinase comprises at least 85% of an amino acid sequence as set forth in SEQ ID NO: 1; the phosphomannomutase comprises at least 85% of an amino acid sequence set forth in SEQ ID NO: 2; the N-acetylhexosamine-1-kinase comprises at least 85% of an amino acid sequence set forth in SEQ ID NO: 3; the mannose-1-phosphate guanyltransferase comprises at least 85% of an amino acid sequence set forth in SEQ ID NO: 4; the GDP-mannose-4,6-dehydratase comprises at least 85% of an amino acid sequence set forth in SEQ ID NO: 5; the GDP-L-fucose-synthase comprises at least 85% of an amino acid sequence set forth in SEQ ID NO: 6; the L-fucokinase comprises at least 85% of an amino acid sequence set forth in SEQ ID NO: 7; the guanosine kinase comprises at least 85% of an amino acid sequence set forth in SEQ ID NO: 8; the polyphosphate kinase comprises any of at least 85% of amino acid sequences set forth in SEQ ID NO: 9 (2D-PPK2), and SEQ ID NO: 14 (PPK3); the pyrophosphatase comprises at least 85% of an amino acid sequence set forth in SEQ ID NO: 10; the guanylate kinase (GMK) comprises at least 85% of an amino acid sequence set forth in SEQ ID NO: 11; the glutamate dehydrogenase comprises at least 85% of an amino acid sequence set forth in SEQ ID NO: 12; the glucose-6-phosphate-dehydrogenase comprises at least 85% of an amino acid sequence set forth in SEQ ID NO: 13; the glucose dehydrogenase comprises at least 85% of an amino acid sequence set forth in SEQ ID NO: 15; the fucosyltransferase comprises at least 85% of an amino acid sequence set forth in SEQ ID NO: 16 (3/4FT).

Also preferably, in any of the above-described inventive methods, the glucokinase comprises at least 90% of an amino acid sequence as set forth in SEQ ID NO: 1; the phosphomannomutase comprises at least 90% of an amino acid sequence set forth in SEQ ID NO: 2; the N-acetylhexosamine-1-kinase comprises at least 90% of an amino acid sequence set forth in SEQ ID NO: 3; the mannose-1-phosphate guanyltransferase comprises at least 90% of an amino acid sequence set forth in SEQ ID NO: 4; the GDP-mannose-4,6-dehydratase comprises at least 90% of an amino acid sequence set forth in SEQ ID NO: 5; the GDP-L-fucose-synthase comprises at least 90% of an amino acid sequence set forth in SEQ ID NO: 6; the L-fucokinase comprises at least 90% of an amino acid sequence set forth in SEQ ID NO: 7; the guanosine kinase comprises at least 90% of an amino acid sequence set forth in SEQ ID NO: 8; the polyphosphate kinase comprises any of at least 90% of amino acid sequences set forth in SEQ ID NO: 9 (2D-PPK2), and SEQ ID NO: 14 (PPK3); the pyrophosphatase comprises at least 90% of an amino acid sequence set forth in SEQ ID NO: 10; the guanylate kinase (GMK) comprises at least 90% of an amino acid sequence set forth in SEQ ID NO: 11; the glutamate dehydrogenase comprises at least 90% of an amino acid sequence set forth in SEQ ID NO: 12; the glucose-6-phosphate-dehydrogenase comprises at least 90% of an amino acid sequence set forth in SEQ ID NO: 13; the glucose dehydrogenase comprises at least 90% of an amino acid sequence set forth in SEQ ID NO: 15; the fucosyltransferase comprises at least 90% of an amino acid sequence set forth in SEQ ID NO: 16 (3/4FT).

More preferably, in any of the above-described inventive methods, the glucokinase comprises at least 95% of an amino acid sequence as set forth in SEQ ID NO: 1; the phosphomannomutase comprises at least 95% of an amino acid sequence set forth in SEQ ID NO: 2; the N-acetylhexosamine-1-kinase comprises at least 95% of an amino acid sequence set forth in SEQ ID NO: 3; the mannose-1-phosphate guanyltransferase comprises at least 95% of an amino acid sequence set forth in SEQ ID NO: 4; the GDP-mannose-4,6-dehydratase comprises at least 95% of an amino acid sequence set forth in SEQ ID NO: 5; the GDP-L-fucose-synthase comprises at least 95% of an amino acid sequence set forth in SEQ ID NO: 6; the L-fucokinase comprises at least 95% of an amino acid sequence set forth in SEQ ID NO: 7; the guanosine kinase comprises at least 95% of an amino acid sequence set forth in SEQ ID NO: 8; the polyphosphate kinase comprises any of at least 95% of amino acid sequences set forth in SEQ ID NO: 9 (2D-PPK2), and SEQ ID NO: 14 (PPK3); the pyrophosphatase comprises at least 95% of an amino acid sequence set forth in SEQ ID NO: 10; the guanylate kinase (GMK) comprises at least 95% of an amino acid sequence set forth in SEQ ID NO: 11; the glutamate dehydrogenase comprises at least 95% of an amino acid sequence set forth in SEQ ID NO: 12; the glucose-6-phosphate-dehydrogenase comprises at least 95% of an amino acid sequence set forth in SEQ ID NO: 13; the glucose dehydrogenase comprises at least 95% of an amino acid sequence set forth in SEQ ID NO: 15; the fucosyltransferase comprises at least 95% of an amino acid sequence set forth in SEQ ID NO: 16 (3/4FT).

Still more preferably, in any of the above-described inventive methods, the glucokinase comprises at least 98% of an amino acid sequence as set forth in SEQ ID NO: 1; the phosphomannomutase comprises at least 98% of an amino acid sequence set forth in SEQ ID NO: 2; the N-acetylhexosamine-1-kinase comprises at least 98% of an amino acid sequence set forth in SEQ ID NO: 3; the mannose-1-phosphate guanyltransferase comprises at least 98% of an amino acid sequence set forth in SEQ ID NO: 4; the GDP-mannose-4,6-dehydratase comprises at least 98% of an amino acid sequence set forth in SEQ ID NO: 5; the GDP-L-fucose-synthase comprises at least 98% of an amino acid sequence set forth in SEQ ID NO: 6; the L-fucokinase comprises at least 98% of an amino acid sequence set forth in SEQ ID NO: 7; the guanosine kinase comprises at least 98% of an amino acid sequence set forth in SEQ ID NO: 8; the polyphosphate kinase comprises any of at least 98% of amino acid sequences set forth in SEQ ID NO: 9 (2D-PPK2), and SEQ ID NO: 14 (PPK3); the pyrophosphatase comprises at least 85% of an amino acid sequence set forth in SEQ ID NO: 10; the guanylate kinase (GMK) comprises at least 98% of an amino acid sequence set forth in SEQ ID NO: 11; the glutamate dehydrogenase comprises at least 98% of an amino acid sequence set forth in SEQ ID NO: 12; the glucose-6-phosphate-dehydrogenase comprises at least 98% of an amino acid sequence set forth in SEQ ID NO: 13; the glucose dehydrogenase comprises at least 98% of an amino acid sequence set forth in SEQ ID NO: 15; the fucosyltransferase comprises at least 98% of an amino acid sequences set forth in SEQ ID NO: 16 (3/4FT).

Most preferably, in any of the above-described inventive methods, the glucokinase comprises an amino acid sequence as set forth in SEQ ID NO: 1; the phosphomannomutase comprises an amino acid sequence set forth in SEQ ID NO: 2; the N-acetylhexosamine-1-kinase comprises an amino acid sequence set forth in SEQ ID NO: 3; the mannose-1-phosphate guanyltransferase comprises an amino acid sequence set forth in SEQ ID NO: 4; the GDP-mannose-4,6-dehydratase comprises an amino acid sequence set forth in SEQ ID NO: 5; the GDP-L-fucose-synthase comprises an amino acid sequence set forth in SEQ ID NO: 6; the L-fucokinase comprises an amino acid sequence set forth in SEQ ID NO: 7; the guanosine kinase comprises an amino acid sequence set forth in SEQ ID NO: 8; the polyphosphate kinase comprises any of amino acid sequences set forth in SEQ ID NO: 9 (2D-PPK2), and SEQ ID NO: 14 (PPK3); the pyrophosphatase comprises an amino acid sequence set forth in SEQ ID NO: 10; the guanylate kinase (GMK) comprises an amino acid sequence set forth in SEQ ID NO: 11; the glutamate dehydrogenase comprises an amino acid sequence set forth in SEQ ID NO: 12; the glucose-6-phosphate-dehydrogenase comprises an amino acid sequence set forth in SEQ ID NO: 13; the glucose dehydrogenase comprises an amino acid sequence set forth in SEQ ID NO: 15; the fucosyltransferase comprises an amino acid sequence set forth in SEQ ID NO: 16 (3/4FT).

### DESCRIPTION OF THE FIGURES

FIG. 1: shows the reaction pathway of the inventive method for producing GDP-fucose, which consists of (a) the formation of fucose-1-phosphate (Fuc-1-P) from L-fucose and ATP, (b) the formation of guanosine triphosphate (GTP) from guanosine and polyphosphate, and (c) the reaction of fucose-1-phosphate with guanosine triphosphate to GDP-fucose.

FIG. 2: shows an exemplary reaction scheme of the inventive method for producing GDP-fucose, which consists of (a) the formation of fucose-1-phosphate (Fuc-1-P) from L-fucose and ATP catalyzed by fucokinase (9), (b) the formation of guanosine triphosphate (GTP) from guanosine and polyphosphate catalyzed by guanosine kinase/inosine kinase (7) and polyphosphate kinase (8), and (c) the reaction of fucose-1-phosphate with guanosine triphosphate to GDP-fucose catalyzed by fucose-1-phosphate guanylyltransferase (9)

FIG. 3: shows an exemplary reaction scheme of the inventive method for producing GDP-fucose, which consists of (a) the formation of mannose-1-phosphate (Man-1-P) from D-mannose and ATP catalyzed by either glucokinase (1) and phosphomannomutase (2) or N-acetylhexosamine-1-kinase (3), (b) the formation of guanosine triphosphate (GTP) from guanosine and polyphosphate catalyzed by guanosine kinase/inosine kinase (7) and polyphosphate kinase (8), and (c) the reaction of mannose-1-phosphate with guanosine triphosphate to GDP-mannose catalyzed by mannose-1-phosphate guanylyltransferase (4) and (d) formation of GDP-4-dehydro-6-deoxy-alpha-D-mannose from GDP-mannose catalyzed by GDP-mannose-4,6-dehydratase (5) and (e) formation of GDP-fucose from GDP-4-dehydro-6-deoxy-alpha-D-mannose and NADPH catalyzed by GDP-L-fucose synthase (6).

FIG. 4A: shows concentration of GDP-fucose prepared by the inventive method at different time points (see Example 3).

FIG. 4B: shows concentration of GDP-fucose prepared by the inventive method using immobilized enzymes at different time points (see example 5).

FIG. 5A: shows a chromatogram of the starting material at t=0.

FIG. 5B: shows a chromatogram of the reaction mixture after 48 hours.

FIG. 6: shows exemplary fucosylated milk saccharides.

FIG. 7: shows amount of bound enzymes on solid support after incubation at 4° C. for 24 hours (see Example 4).

FIG. 8: shows amount of formed GDP-fucose after reaction with the immobilized enzymes for 24 hours at 30° C. (see Example 4).

FIG. 9: shows the exemplary reaction scheme of the inventive method for producing L-fucose from guanosine and D-mannose, which consists of (a) the formation of mannose-1-phosphate (Man-1-P) from D-mannose and ATP catalyzed by either glucokinase (1) and phosphomannomutase (2) or N-acetylhexosamine-1-kinase (3), (b) the formation of guanosine triphosphate (GTP) from guanosine and polyphosphate catalyzed by guanosine kinase/inosine kinase (7) and polyphosphate kinase (8), and (c) the reaction of mannose-1-phosphate with guanosine triphosphate to GDP-mannose catalyzed by mannose-1-phosphate guanylyltransferase and (d) formation of GDP-4-dehydro-6-deoxy-alpha-D-mannose from GDP-mannose catalyzed by GDP-mannose-4,6-dehydratase and (e) formation of GDP-fucose from GDP-4-dehydro-6-deoxy-alpha-D-mannose and NADPH catalyzed by GDP-L-fucose synthase and formation of L-fucose from GDP-L-fucose catalyzed by type 1 galactoside alpha-(1,2)-fucosyltransferase in absence of an acceptor molecule.

FIG. 10: shows an exemplary reaction scheme of the inventive method for producing 2′-fucosyllactose from guanosine and D-mannose, which consists of (a) the formation of mannose-1-phosphate (Man-1-P) from D-mannose and ATP catalyzed by either glucokinase (1) and phosphomannomutase (2) or N-acetylhexosamine-1-kinase (3), (b) the formation of guanosine triphosphate (GTP) from guanosine and polyphosphate catalyzed by guanosine kinase/inosine kinase (7) and polyphosphate kinase (8), and (c) the reaction of mannose-1-phosphate with guanosine triphosphate to GDP-mannose catalyzed by mannose-1-phosphate guanylyltransferase and (d) formation of GDP-4-dehydro-6-deoxy-alpha-D-mannose from GDP-mannose catalyzed by GDP-mannose-4,6-dehydratase and (e) formation of GDP-fucose from GDP-4-dehydro-6-deoxy-alpha-D-mannose and NADPH catalyzed by GDP-L-fucose synthase and formation of 2′-fucosyllactose from GDP-L-fucose and lactose catalyzed by type 1 galactoside alpha-(1,2)-fucosyltransferase.

FIG. 11: shows the reaction cascade from (A1) GDP-mannose to GDP-fucose performed in Example 6-A1; (A2) GDP-mannose to GDP-fucose in presence of glucose and G6PDH as performed in Example 6-A2; and (A3) mannose and GTP to GDP-fucose in presence of glutamate and GLDH as performed in Example 6-A3. Reactions were conducted to demonstrate that NADPH recycling can drive the multi-enzyme reaction equilibrium towards the side of GDP-fucose.

FIG. 12: shows chromatogram of reaction aliquots of reactions A1, A2 and A3 after overnight incubation). The chromatogram (A1) shows that the reaction equilibrium is on the side towards GDP-mannose. When NADPH recycling is implemented the reaction equilibrium can be driven towards GDP-fucose (see A2 and A3).

FIG. 13: shows the reaction cascade from the reaction cascade from mannose and guanosine to GDP-fucose as performed in Example 7.

FIG. 14: shows reaction time course of the reaction in example 7 as measured by ion chromatography.

FIG. 15: shows the reaction cascade from mannose and guanosine to GDP-fucose as performed in Example 8.

FIG. 16: shows reaction time course of the reaction in example 8 as measured by ion chromatography.

FIG. 17: shows the reaction cascade from mannose and guanosine to 3-fucosyl-lactose as performed in Example 9.

FIG. 18: shows synthesis of 3-fucosyllactose from mannose and guanosine. Reaction chromatogram of a reaction aliquot taken after a reaction time of 48 hours (black) and of 3-fucosyllactose standard samples. The peaks were detected by amperometric detection.

FIG. 19: shows synthesis of 3-fucosyllactose from guanosine and fucose (reaction D2 of example 9). Chromatogram of a reaction aliquot taken after a reaction time of 48 hours. The peaks were detected by amperometric detection.

FIG. 20: shows the production of L-fucose from D-mannose. The reaction of sample in example 8 was heated for 1 hour at 95° C. (see, example 10). An aliquot of the heated sample was taken and measured by ion chromatography with amperometric detection (black) and compared against a fucose standard (Fuc).

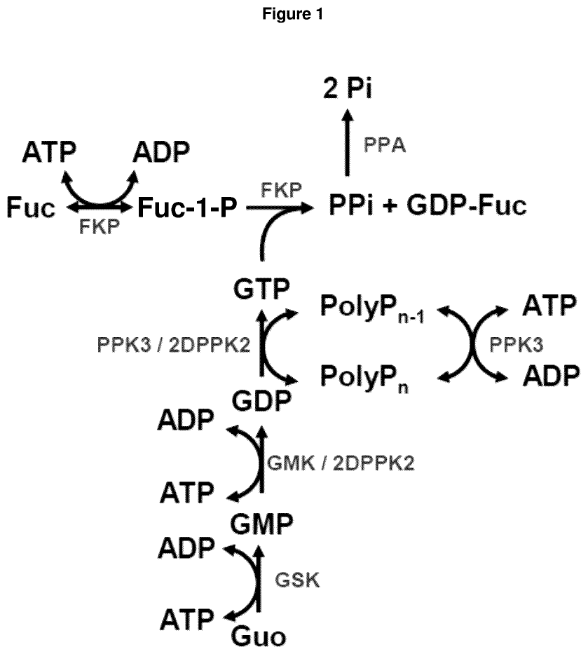

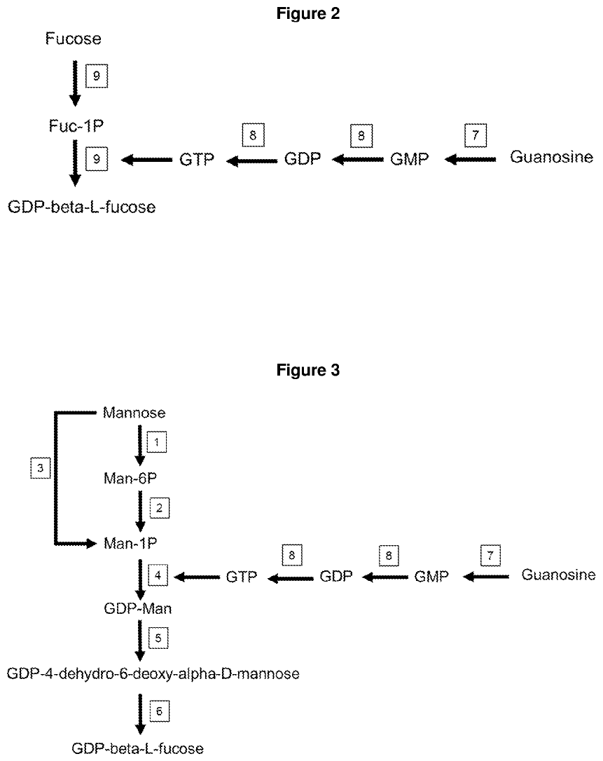

The following examples are included to demonstrate preferred embodiments of the invention. It should be appreciated by those skilled in the art that the techniques disclosed in the examples, which follow represent techniques discovered by the inventor to function well in the practice of the invention, and thus can be considered to constitute preferred modes for its practice. However, those skilled in the art should, in light of the present disclosure, appreciate that many changes can be made in the specific embodiments, which are disclosed and still obtain a like or similar result without departing from the spirit and scope of the invention.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### EXAMPLES

### Abbreviations and Acronyms

ADP adenosine 5′-diphosphate
ATP adenosine 5′-triphosphate
Fuc L-fucose
GSK guanosine kinase
GDP guanosine 5′-diphosphate
GDH glucose dehydrogenase; glucose-1-dehydrogenase
GLDH glutamate dehydrogenase
G6PDH glucose-6-phosphate-dehydrogenase
GMD GDP-D-mannose-4,6-dehydratase
GMK guanylate kinase
GMP guanosine 5′-monophosphate
GLK glucokinase
GTP guanosine 5′-triphosphate
GUO guanosine
Lac D-lactose
Man D-mannose
ManB phosphomannomutase
ManC mannose-1-phosphate guanyltransferase
NADP nicotinamide adenine dinucleotide phosphate
NADPH reduced nicotinamide adenine dinucleotide phosphate
NAHK N-acetylhexosamine-1-kinase
PolyP polyphosphate
PPi pyrophosphate
Pi phosphate
PPK2 polyphosphate kinase 2
PPK3 polyphosphate kinase 3
2D-PPK2 2-domain polyphosphate kinase 2
FKP L-fucokinase/L-fucose-1-phosphate guanylyltransferase
PmPpA Pasteurella multocida inorganic pyrophosphatase (PPA)
WCAG GDP-4-keto-6-deoxymannose-3,5-epimerase-4-reductase
3/4FT α-1-3/4-fucosyltransferase

Chemicals & Reagents

Unless otherwise stated, all chemicals and reagents were acquired from Sigma-Aldrich, and were of the highest purity available. Solid supports were obtained from Resindion, ChiralVision, Röhm GmbH & Co. KG and micromod GmbH.

### Example 1: Preparation of Enzymes

The genes encoding for the enzymes GSK, PPK2, FKP and PmPpA were cloned into standard expression vectors as listed in Table 1. The expression vectors were transformed into E. coli BL21 Gold (DE3).

TABLE 1

| | | | | Expression |
|---|---|---|---|---|
| Enzyme | Source | Plasmid | Inducer | host |
| guanosine kinase | Exiguobacterium | pET-28a(+) | IPTG | E. coli BL21 |
| (GSK) | acetylicum | | | Gold (DE3) |
| polyphosphate kinase | Pseudomonas | pET-28a(+) | IPTG | E. coli BL21 |
| (2D-PPK2) | aeruginosa | | | Gold (DE3) |
| L-fucokinase/L-fucose-1- | Bacteroides | pET-100/D- | IPTG | E. coli BL21 |
| phosphate- | fragilis | TOPO | | Gold (DE3) |
| guanylyltransferase (FKP) | | | | |
| inorganic pyrophosphatase | Pasteurella | pET-28a(+) | IPTG | E. coli BL21 |
| (PmPpA) | multocida | | | Gold (DE3) |

Transformants were grown in 1 L shaking flasks with baffles in a volume of 500 ml of LB medium (lysogeny broth) supplemented with 50 μg/ml Kanamycin. The cultures were grown at 37° C. up to OD₆₀₀=0.8. The expression was induced by addition of IPTG with a final concentration of 0.5 mM to the culture. Expression time was terminated after 12-18 hours at 20° C. Biomass was separated from the medium by centrifugation at 6,000×g for 10 min. Successful expression of the respective enzyme was analyzed by SDS-PAGE following standard operating procedures (Laemmli, Nature 1970, 227, 680-685). The wet biomass was stored at −20° C.

For purification, typically 30 ml of equilibration buffer were added to 3 g of frozen biomass. The equilibration buffer contains cOmplete™ protease inhibitor cocktail at pH 7.5: 100 mM HEPES (4-(2-hydroxyethyl)-1-piperazineethanesulfonic acid), 10 mM MgCl₂, 5 mM MnSO₄, 300 mM NaCl and 5 vol % glycerol. Following thawing at 4° C. under stirring, cells were disrupted by four passages through a high pressure homogenizer (Emulsiflex C5, Avestin Inc., Ottawa, Canada) at 1,000 bar with intermediate cooling on ice. After centrifugation (45 min, 20,000×g), the supernatant was applied to an equilibrated Immobilized Metal Affinity Chromatography (IMAC) column (10 ml CV) containing Ni²⁺ Sepharose™ High Performance chromatography material from Amersham Biosciences (Uppsala, Sweden). Unbound proteins were washed out using equilibration buffer. Immobilized protein was eluted in 1 ml fractions using elution buffer. Finally, the enzyme solutions were concentrated by Centrifugal Filter Units Amicon® Ultra-15 with a 50 kDa cut-off from Merck Millipore (Darmstadt, Germany). No enzyme loss was observed during the ultrafiltration. The enzymes were stored in 50% glycerol at −20° C. The protein concentration was determined by Bradford assay using BSA as standard (Bradford, Analytical Biochemistry 1976, 72(1), 248-254).

### Example 2: Homogeneous Preparation of GDP-Fucose

The purified enzymes (Table 1) are mixed together with guanosine, L-fucose, ATP, polyphosphate, and HEPES buffer to the concentrations as listed in Table 2. Experiments conducted in low binding protein vials. Guanosine was solubilized in 6 vol % dimethyl sulfoxide (DMSO) prior addition to the mixture. The reaction was carried out at 30° C. in a thermomixer.

TABLE 2

| Reactants | Concentration [mmol/L] |
|---|---|
| guanosine | 2 |
| polyphosphate (n = 14 or 25) | 4 |
| L-fucose | 2 |
| ATP | 2 |
| HEPES Buffer | 50 |
| Ma²⁺ | 2.5 |
| Mn²⁺ | 2.5 |
| Enzyme | Concentration [mg/mL] |
| GSK | 0.3 |
| 2D-PPK2 | 0.7 |
| FKP | 0.2 |
| PmPpA | 0.2 |

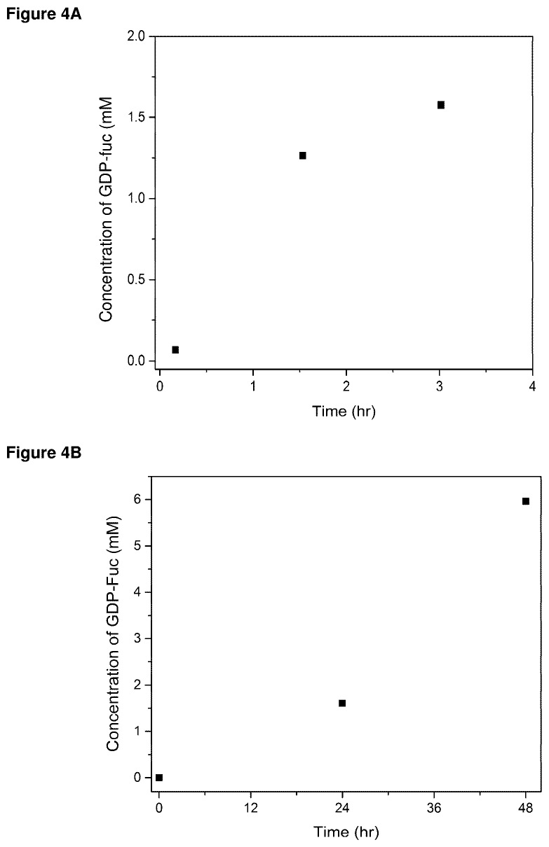

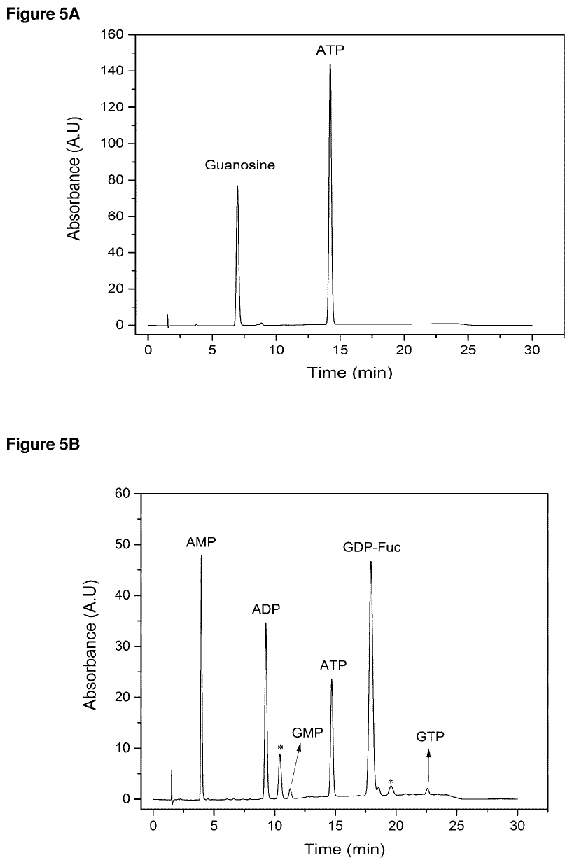

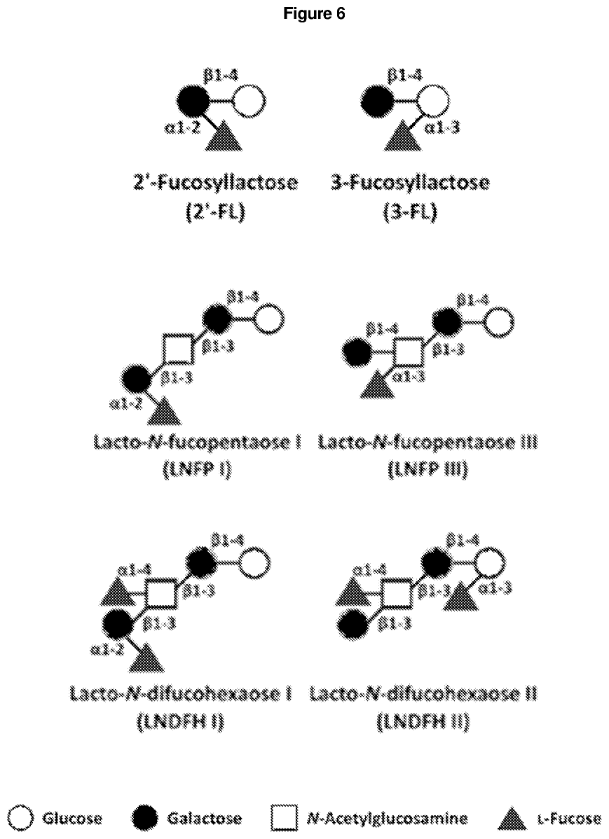

After almost three hours 90% conversion of substrate (guanosine) to GDP-fucose was obtained (See FIG. 4A). No other end-products were detected (See FIG. 5B showing a chromatogram of the reaction mixture after 48 hours).

### Example 3: Failed Synthesis of GDP-Fucose

The synthesis of GDP-fucose was carried out as described in Example 2, but without adding DMSO. The reaction was carried out at 30° C. in a thermomixer and the obtained reaction mixture remained turbid. After three hours no conversion of substrate (guanosine) to GDP-fucose was observed.

This Example demonstrates that the mere combination of the two enzymatic pathways does not provide GDP-fucose.

### Example 4: Immobilization of Enzymes on Solid Support

Enzymes were immobilized on the solid supports in order to allow the multiple use of the enzymes.

Cell lysates obtained in Example 1 by high pressure homogenization were centrifuged and filtered to remove cell debris. The resins: sepabeads (Resindion): EC-EP, EP403/M, EC-HFA, EC-EA/M and EC-HA; immobeads (ChiralVision) IB-COV1, IB-COV2, IB-COV3, IB-AN11, IB-AN12, IB-AN13, IB-AN14, IB-CAT1, IB-ADS1, IB-ADS2, IB-ADS3 and IB-ADS4; Eupergit (Röhm GmbH & Co. KG) and magnetic particles (micromod GmbH): Nano-mag, Sicastar-6 and Sicastar-1.5 wer incubated together with the enzymes for 24 hours at 4° C.

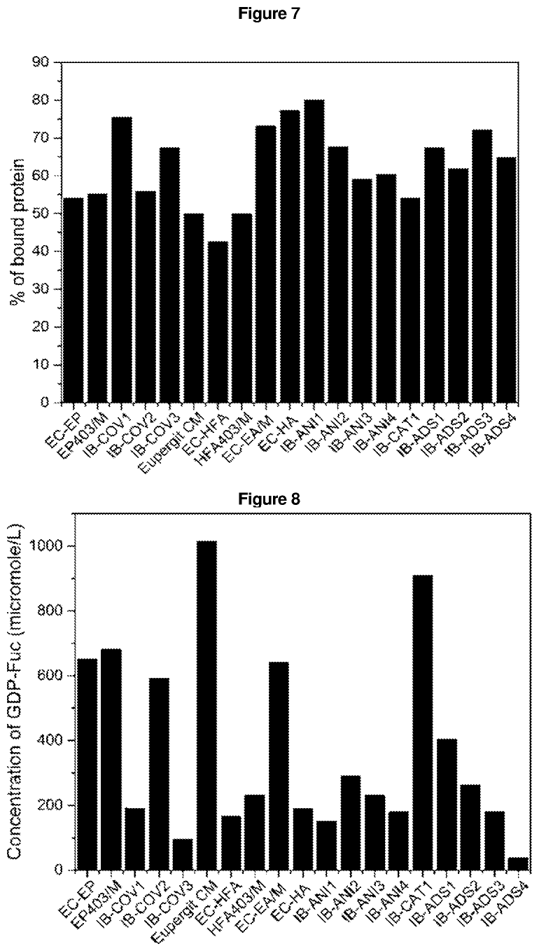

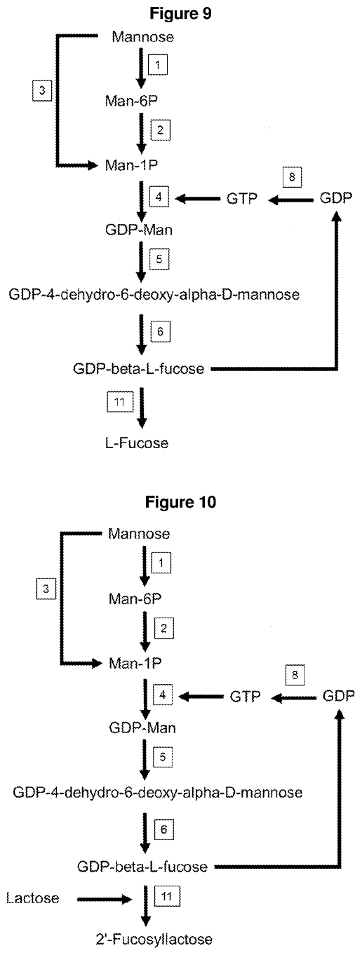

The protein assay was done by BCA assay. Results of total bound protein are shown in FIG. 7.

After immobilization the enzyme loaded resins were washed with buffer as described in Example 1. The resins were incubated with a solution of reactants as shown in Table 3 at 30° C. for 24 hours.

TABLE 3

| Reactants | Concentration [mmol/L] |
|---|---|
| guanosine | 3.9 (4 vol % DMSO) |
| polyphosphate (n = 25) | 12.5 |
| L-fucose | 5.3 |
| ATP | 12 |
| HEPES Buffer | 120 |
| MgCl₂ | 8 |
| MnCl₂ | 8 |
| NaCl | 200 |

The formation of GDP-fucose was observed for all loaded resins, as shown in FIG. 8.

### Example 5: Heterogeneous Preparation of GDP-Fucose on Magnetic Particles

To this extent, fermentation broths (see Example 1) of 135 mL of FKP, 45 mL of PmPpA, 90 mL GSK and 90 mL of 2D-PPK2 were combined and lysed in 25 mL buffer (as described in Example 1) containing 10 mM imidazole, to obtain 0.5 mL of an enzyme mixture, which was incubated with 12.5 mg of Sicastar-6 magnetic resins. After 1 hour of incubation at 10° C., resins were washed with buffer and combined in a vial with 0.25 mL of a solution of reagents (see Table 4). The mixture was incubated at 30° C. for 48 hours.

TABLE 4

| Reactants | Concentration [mmol/L] |
|---|---|
| guanosine | 6 (4 vol % DMSO) |
| polyphosphate (n = 25) | 9.3 |
| L-fucose | 6 |
| ATP | 9.6 |
| HEPES Buffer | 50 |
| MgCl₂ | 15 |
| MnCl₂ | 5 |
| NaCl | 30 |
| KCl | 30 |

After 48 hours nearly quantitative conversion (98%) of substrate (guanosine) to GDP-fucose was obtained (See FIG. 4B).

### Example 6: Recycling of NADPH

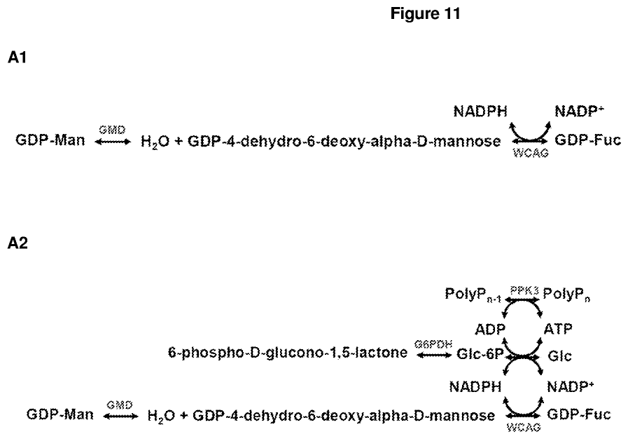

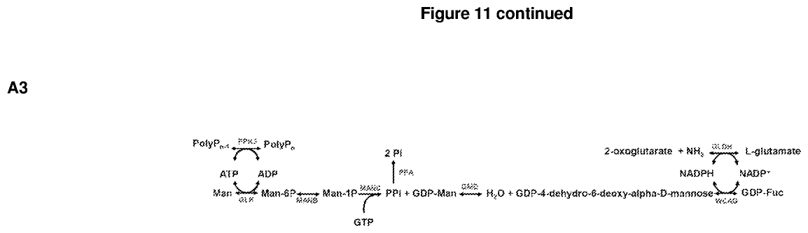

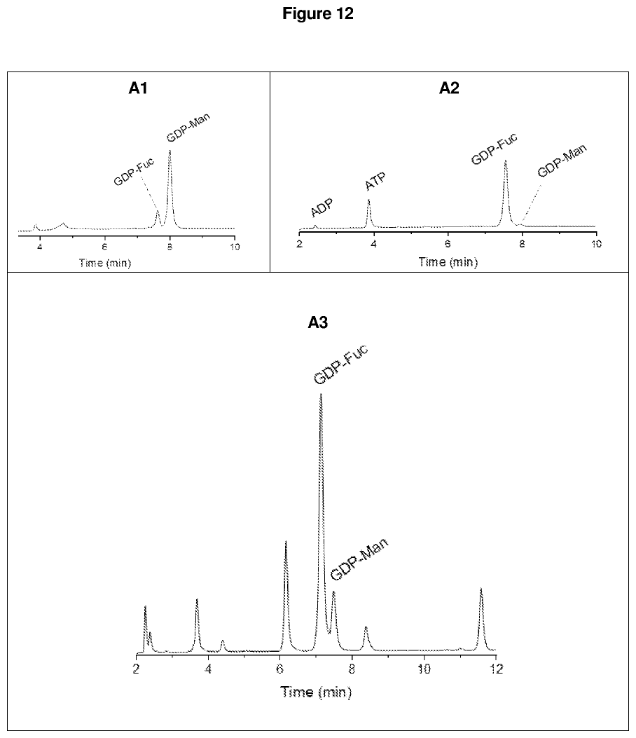

A one-pot reaction was conducted (see reaction conditions of reaction A1 in Table 5) to show that the equilibrium of the GDP-mannose 4,6-dehydratase (GMD, E.C. 4.2.1.47) and GDP-L-fucose synthase (wcaG, E.C. 1.1.1.271) catalyzed reactions from GDP-mannose to GDP-fucose is on the side of GDP-mannose (see FIGS. 11-A1 and 12-A1). In this experiment, first the enzymes were dispensed onto a vial up to the amounts mentioned in table 5. Buffer, co-factor, GDP-Man, NADPH were added in thus order, up to the mentioned concentration in table 5. The reaction was performed in 1.5 mL Eppendorf vials, at 37° C. and 550 rpm in the Eppendorf thermomixer comfort. Aliquots were taken at different time points and quenched by heating at 90° C. for 3 minutes and measured by ion exchange chromatography. It could be seen that the equilibrium is on the side of GDP-Man (see FIG. 12-A1).

To push the equilibrium of the reaction towards GDP-Fuc, coupling to another reaction (in a way that NADP⁺ is constantly removed) needs to be engineered. In reaction A2 the enzymes Glk, PPK3 and glucose-6-phosphate-dehydrogensase (purchased from Merck—10165875001) (G6PDH, E.C: 1.1.1.49) were used to recycle NAPDH and increase the GDP-fucose yield. In this experiment, first the enzymes were dispensed into a vial up to the amounts mentioned in the table 6. After addition of enzymes, buffer, co-factor, GDP-Man, NADPH, glucose, ATP and polyphosphate were added in this order, up to the mentioned concentrations in table 6. The reaction was performed in a 1.5 mL Eppendorf vial, at 37° C. and 550 rpm in the Eppendorf thermomixer Comfort. Aliquots were taken at different time points and quenched by heating at 90° C. for 3 minutes and then measured by ion exchange chromatography. For the recycling the inexpensive substrates glucose and polyphosphate are used as substrates (see FIGS. 11-A2 and 12-A2).

Another experiment was performed for the production of GDP-Fuc from Man, ATP, GTP and polyphosphate, and L-glutamate for the regeneration of NADPH and shifting the equilibrium towards GDP-Fuc. In this experiment, first enzymes were dispensed into a vial up to the amounts mentioned in the table 7. After addition of enzymes, buffer, co-factor, Man, GTP, ATP and L-glutamate, and polyphosphate were added in this order, up to the mentioned concentrations in table 7. The reaction was performed in a 1.5 mL Eppendorf vial, at 37° C. and 550 rpm in the Eppendorf thermomixer Comfort. Aliquots were taken at different time points and quenched by heating at 90° C. for 3 minutes and then measured by ion exchange chromatography. In reaction A3 (see reaction conditions of reaction A3 in Table 7) glutamate dehydrogenase (purchased from Merck—10197734001) (GLDH, E.C. 1.4.1.4) was used to recycle NADPH from L-glutamate and water. (see FIGS. 11-A3 and 12-A3).

TABLE 5
Reactions conditions of examples A1.

| Enzymes | Concentration | |
|---|---|---|
| WCAG | 1.03 | μg/μL |
| GMD | 1.75 | μg/μL |
| Substrates | | |
| GDP-Man | 2 | mM |
| NADPH | 2 | mM |
| Buffer and co-factor | | |
| Tris-HCl (pH = 8) | 100 | mM |
| MgCl₂ | 10 | mM |
| Volume | 40 | μL |

TABLE 6
Reaction conditions for reaction A2.

| Enzymes | Concentration | |
|---|---|---|
| WCAG | 0.8 | μg/μL |
| GMD | 1.4 | μg/μL |
| GLK | 0.129 | μg/μL |
| PPK3 | 0.07 | μg/μL |
| G6PDH | 0.28 | μg/μL |
| Substrates | | |
| GDP-Man | 4 | mM |
| NADPH | 0.5 | mM |
| Glucose | 10 | mM |
| ATP | 1 | mM |
| PolyP₂₅ | 2 | mM |
| Buffer and co-factor | | |
| Tris-HCl (pH = 8) | 100 | mM |
| MgCl₂ | 10 | mM |
| Volume | 50 | μL |

TABLE 7
Reaction conditions of reaction A3.

| Enzymes | Concentrations | |
|---|---|---|
| WCAG | 0.3 | μg/μL |
| GMD | 0.52 | μg/μL |
| GLK | 0.09 | μg/μL |
| PPK3 | 0.27 | μg/μL |
| GLDH | 2.99 | μg/μL |
| ManB/C | 0.046 | μg/μL |
| PPA | 0.047 | μg/μL |
| Substrates | | |
| Man | 20 | mM |
| GTP | 20 | mM |
| ATP | 2 | mM |
| PolyP₂₅ | 5 | mM |
| L-glutamate | 50 | mM |
| NADPH | 1 | mM |
| Buffer and co-factor | | |
| Tris-HCl (pH = 9) | 150 | mM |
| MgCl₂ | 50 | mM |
| Volume | 200 | μL |

### Example 7: Synthesis of GDP-Fucose from D-Mannose Using Glk

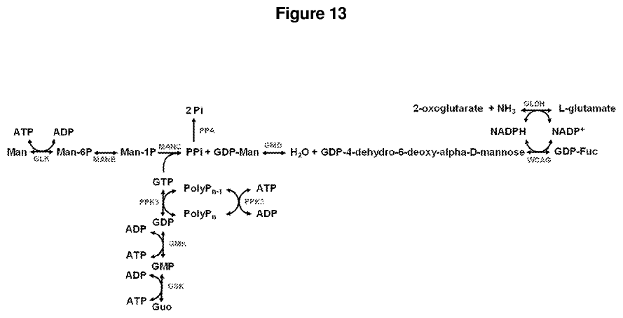

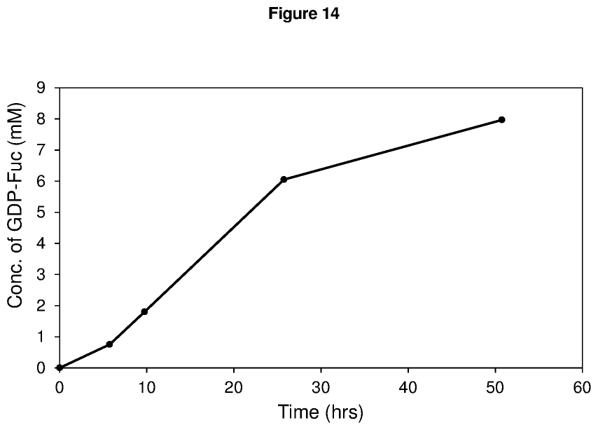

A one-pot enzymatic reaction was conducted to validate GDP-fucose synthesis from mannose through the cascade shown in FIG. 13. In this experiment, first enzymes were dispensed into a vial up to amounts mentioned in the table 8. After addition of enzymes, buffer, co-factor, mannose, ATP, L-glutamate, NADPH, guanosine (from a stock containing DMSO) and polyphosphate were added in this order, up to the mentioned concentrations in table 8. The reaction was performed in a 1.5 mL Eppendorf vial, at 37° C. and 550 rpm in a Eppendorf thermomixer Comfort. Aliquots were taken at different time points and quenched by heating at 90° C. for 3 minutes and then measured by ion exchange chromatography. Reaction time course of the reaction in example 7 is measured by ion chromatography as shown in FIG. 14.

TABLE 8
Reaction conditions as used in example 7.

| Enzymes | Concentrations | |
|---|---|---|
| GSK | 0.128 | μg/μL |
| GMK | 0.034 | μg/μL |
| PPK3 | 0.2 | μg/μL |
| GLK | 0.11 | μg/μL |
| MANB/C | 0.071 | μg/μL |
| WCAG | 0.24 | μg/μL |
| GMD | 0.4 | μg/μL |
| GLDH | 3.45 | μg/μL |
| PPA | 0.04 | μg/μL |
| Substrates | | |
| Man | 11.5 | mM |
| Guanosine (in DMSO) | 11.5 | mM |
| ATP | 2.9 | mM |
| PolyP₂₅ | 5.17 | mM |
| L-glutamate | 86 | mM |
| NADPH | 2.8 | mM |
| Buffer and co-factor | | |
| Tris-HCl (pH = 8) | 115 | mM |
| MgCl₂ | 29 | mM |
| Volume | 175 | μL |

### Example 8: Synthesis of GDP-Fucose from D-Mannose Using NahK

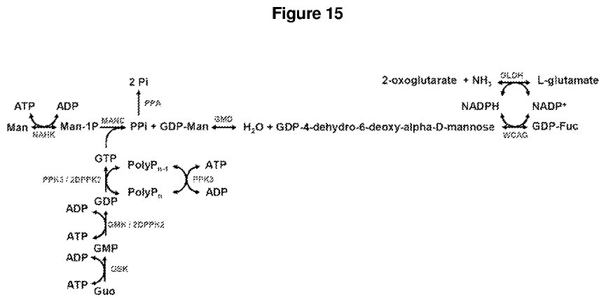

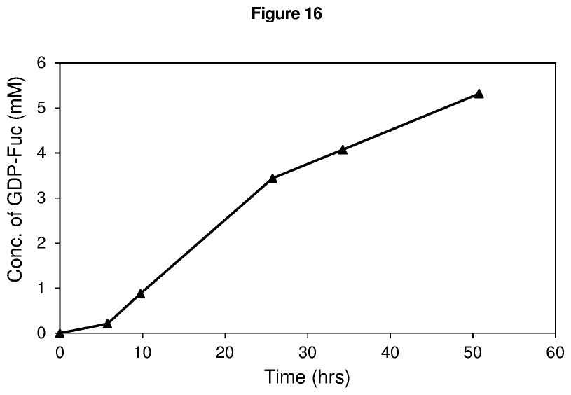

A one-pot enzymatic reaction was conducted to validate GDP-fucose synthesis from mannose through the cascade shown in FIG. 15. In this experiment, first enzymes were dispensed into a vial up to amounts mentioned in the table 9. After addition of enzymes, buffer, co-factor, mannose, ATP, L-glutamate, NADPH, guanosine (stock was prepared in DMSO) and polyphosphate were added in the order, up to the mentioned concentrations in table 9. The reaction was performed in 1.5 mL Eppendorf vials, at 37° C. and 550 rpm in a Eppendorf thermomixer comfort. Aliquots were taken at different time points and quenched by heating at 90° C. for 3 minutes and then measured by ion exchange chromatography. Reaction time course of the reaction in example 8 is measured by ion chromatography as shown in FIG. 16.

TABLE 9
Reaction conditions for example 8.

| Enzymes | Concentration | |
|---|---|---|
| GSK | 0.12 | μg/μL |
| GMK | 0.03 | μg/μL |
| PPK3 | 0.19 | μg/μL |
| NAHK | 0.14 | μg/μL |
| MANB/C | 0.06 | μg/μL |
| WCAG | 0.22 | μg/μL |
| GMD | 0.38 | μg/μL |
| GLDH | 3.26 | μg/μL |
| PPA | 0.03 | μg/μL |
| Substrates | | |
| Man | 10.9 | mM |
| Guanosine (in DMSO) | 10.9 | mM |
| ATP | 2.75 | mM |
| PolyP₂₅ | 4.9 | mM |
| L-glutamate | 81 | mM |
| NADPH | 2.6 | mM |
| Buffer and co-factor | | |
| Tris-HCl (pH = 8) | 108 | mM |
| MgCl₂ | 27 | mM |
| Volume | 185 | μL |

### Example 9: Synthesis of 3-fucosyllactose

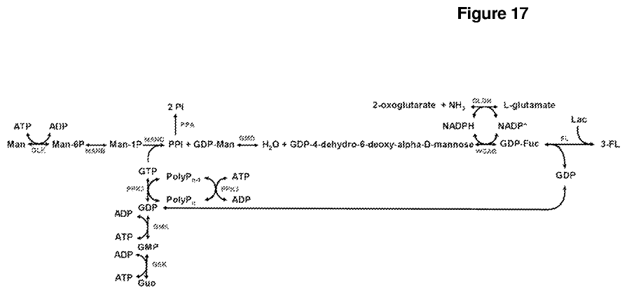

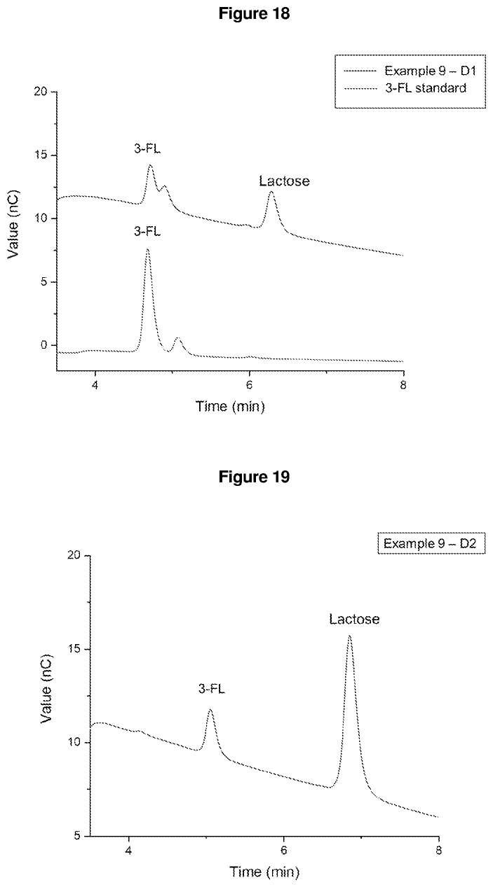

The cascades for GDP-fucose production can be coupled to fucosyltransferases to fucosylate molecules or biomolecules, e.g. human milk oligosaccharides and therapeutic proteins. The coupling is performed in one-pot reactions (see FIG. 17). Reactions were conducted where the GDP-fucose cascade was coupled to 3/4-fucosyltransferase to produce 3-fucosyllactose starting from D-mannose (reaction D1; see FIG. 18). In this experiment, first enzymes were dispensed into a vial up to amounts mentioned in the table 10. After addition of enzymes, buffer, co-factor, mannose, ATP, L-glutamate, NADPH, lactose, guanosine (stock was prepared in DMSO) and polyphosphate were added in this order, up to the mentioned concentrations in table 10. The reaction was performed in 1.5 mL Eppendorf vials, at 37° C. and 550 rpm in a Eppendorf thermomixer comfort. Aliquot was taken and quenched by heating at 90° C. for 3 minutes and then measured by ion exchange chromatography. Another experiment was performed to produce 3-fucosyllactosein which GDP-Fuc was produced from guanosine, L-fucose (reaction D2; see FIG. 19). In this experiment, first enzymes added up to amounts mentioned in the table 11. After addition of enzymes, buffer, co-factor, L-fucose, ATP, lactose, guanosine (stock was prepared in DMSO) and polyphosphate were added in the order, up to the mentioned concentration in table 11. The reaction was performed in 1.5 mL Eppendorf vials, at 37° C. and 550 rpm in a Eppendorf thermomixer comfort. Aliquot was taken and quenched by heating at 90° C. for 3 minutes and then measured by ion exchange chromatography.

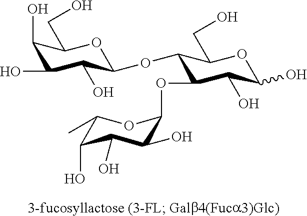

TABLE 10
Reaction conditions for reaction D1.

| Enzymes | Concentration | |
|---|---|---|
| GSK | 0.128 | μg/μL |
| GMK | 0.034 | μg/μL |
| PPK3 | 0.2 | μg/μL |
| GLK | 0.11 | μg/μL |
| MANB/C | 0.071 | μg/μL |
| WCAG | 0.24 | μg/μL |
| GMD | 0.4 | μg/μL |
| GLDH | 3.45 | μg/μL |
| PPA | 0.04 | μg/μL |
| 3/4FT | 0.07 | μg/μL |
| Substrates | | |
| Man | 8.7 | mM |
| Guanosine (in DMSO) | 2.9 | mM |
| ATP | 2.9 | mM |
| PolyP₂₅ | 5.2 | mM |
| L-glutamate | 29 | mM |
| NADPH | 1.45 | mM |
| Lactose | 11.6 | mM |
| Buffer and co-factor | | |
| Tris-HCl (pH = 8) | 116 | mM |
| MgCl₂ | 43 | mM |
| Volume | 172 | μL |

TABLE 11
Reaction conditions for reaction D2.

| Enzymes | Concentration | |
|---|---|---|
| GSK | 0.16 | μg/μL |
| GMK | 0.042 | μg/μL |
| PPK3 | 0.25 | μg/μL |
| FKP | 0.42 | μg/μL |
| PPA | 0.05 | μg/μL |
| 3/4FT | 0.07 | μg/μL |
| Substrates | | |
| Fucose | 14.1 | mM |
| Guanosine (in DMSO) | 3.5 | mM |
| ATP | 3.6 | mM |
| PolyP₂₅ | 6.3 | mM |
| Lactose | 14.1 | mM |
| Buffer and co-factor | | |
| Tris-HCl (pH = 8) | 141 | mM |
| MgCl₂ | 35 | mM |
| Volume | 141 | μL |

### Example 10: Production of L-Fucose from D-Mannose

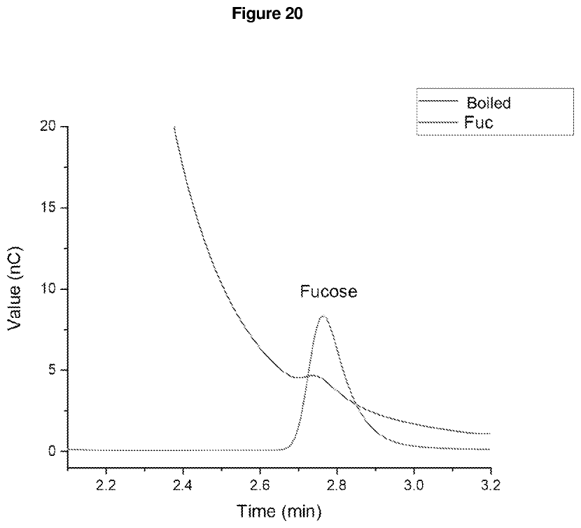

The cascade described in examples 7 and 8 can be used to produce L-fucose from D-mannose and guanosine. The reaction in example 8 was heated at 95° C. for 1 hour. An aliquot taken after heating was measured by ion chromatography (see FIG. 20).

A sequence listing is attached to this application comprising the sequences of the following table:

| SEQ ID | description |
|---|---|
| 1 | Glucokinase |
| 2 | Phosphomannomutase |
| 3 | N-acetylhexosamine-1-kinase |
| 4 | Mannose-1-phosphate guanylyltransferase |
| 5 | GDP-mannose 4,6-dehydratase |
| 6 | GDP-L-fucose synthase |
| 7 | L-fucokinase |
| 8 | Guanosine kinase |
| 9 | 2-domain polyphosphate kinase 2 |
| 10 | inorganic pyrophosphatase |
| 11 | Guanylate kinase |
| 12 | Glutamate dehydrogenase 1 (GLDH) |
| 13 | Glucose-6-phosphate 1-dehydrogenase (G6PDH) |
| 14 | phosphotransferase 3 (PPK3) |
| 15 | Glucose/galactose 1-dehydrogenase (GDH) |
| 16 | alpha-1,3/4-fucosyltransferase (3/4FT) |

1.-19. (canceled)

20. A method for producing guanosine 5′-diphospho-β-L-fucose from guanosine and D-mannose comprising the following steps: 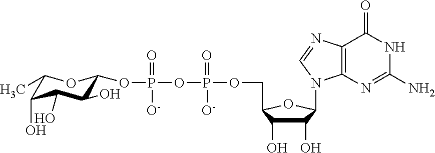
A) providing a solution comprising
(i) guanosine and D-mannose represented by the following formulae 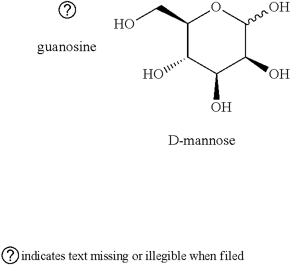 [SMILES: OC[C@H]1OC(O)[C@@H](O)[C@@H](O)[C@@H]1O]
(ii) polyphosphate, adenosine triphosphate, a co-solvent for solubilizing guanosine and NADPH; and
providing a set of enzymes comprising a guanosine kinase, a polyphosphate kinase, a mannose-1-phosphate guanylyltransferase, a GDP-mannose-4,6-dehydratase, a GDP-L-fucose-synthase and either (a) a glucokinase and a phosphomannomutase, or (b) an N-acetylhexosamine-1-kinase.
B) producing guanosine 5′-diphospho-β-L-fucose from guanosine and D-mannose in the presence of the set of enzymes, polyphosphate, adenosine triphosphate, NADPH and the co-solvent.

21. The method according to claim 20, wherein the set of enzymes further comprises a pyrophosphatase.

22. The method according to claim 20, wherein the set of enzymes is co-immobilized on a solid support.

23. The method according to claim 22, wherein the set of enzymes is immobilized on a solid support from cell lysate or the set of enzymes is co-immobilized on a solid support from cell lysate.

24. The method according to claim 20, wherein the concentration of guanosine and D-mannose in the solution provided in step A) is in the range of 0.2 mM to 5,000 mM.

25. The method according to claim 20, wherein the polyphosphate is a long-chain polyphosphate having at least 25 phosphate residues.

26. The method according to claim 20, wherein the guanosine 5′-diphospho-β-L-fucose is produced in a single reaction mixture.

27. The method according to claim 20, wherein the amount of co-solvent is from 0.01 vol % to 30 vol % based on total volume of the solution provided in step A).

28. The method according to claim 20, wherein the co-solvent is dimethyl sulfoxide.

29. The method according to claim 20, wherein the method further comprises the step
producing L-fucose from guanosine 5′-diphospho-β-L-fucose in the presence of a fucosyltransferase and in the absence of an acceptor; or
producing L-fucose by heating the guanosine 5′-diphospho-β-L-fucose at the temperature in a range of 80 to 100° C.

30. The method according to claim 20, further comprising the step of
isolating the guanosine 5′-diphospho-β-L-fucose.

31. The method according to claim 20, further comprising the step of
producing a fucosylated saccharide, fucosylated glycopeptide, fucosylated glycoprotein fucosylated protein, fucosylated peptide or small molecule from guanosine 5′-diphospho-β-L-fucose and a saccharide, glycopeptide, glycoprotein, protein, peptide or small molecule by forming an O-glycosidic bond between guanosine 5′-diphospho-β-L-fucose and an available hydroxyl group of the saccharide, glycopeptide glycoprotein, protein, peptide or small molecule in the presence of a fucosyltransferase.

32. The method according to claim 20, wherein the set of enzymes further comprises any one of a glucose dehydrogenase, a glucose-6-phosphate dehydrogenase and a glutamate dehydrogenase.

33. The method according to claim 32, wherein the set of enzymes further comprises a guanylate kinase.

34. The method according to claim 20, wherein the guanosine kinase comprises an amino acid sequence having at least 95% sequence identity to SEQ ID NO: 8; the polyphosphate kinase comprises an amino acid sequence having at least 95% sequence identity to SEQ ID NO: 9 or SEQ ID NO. 14; the mannose-1-phosphate guanylyltransferase comprises an amino acid sequence having at least 95% sequence identity to SEQ ID NO: 1, the GDP-mannose-4,6-dehydratase comprises an amino acid sequence having at least 95% sequence identity to SEQ ID NO: 5, the GDP-L-fucose-synthase comprises an amino acid sequence having at least 95% sequence identity to SEQ ID NO: 6, the glucokinase comprises an amino acid sequence having at least 95% sequence identity to SEQ ID NO: 1, the phosphomannomutase comprises an amino acid sequence having at least 95% sequence identity to SEQ ID NO: 2, and the N-acetylhexosamine-1-kinase comprises an amino acid sequence having at least 95% sequence identity to SEQ ID NO: 3.

35. The method according to claim 34, wherein the guanosine kinase comprises an amino acid sequence of SEQ ID NO: 8; the polyphosphate kinase comprises an amino acid sequence of SEQ ID NO: 9 or SEQ ID NO. 14; the mannose-1-phosphate guanylyltransferase comprises an amino acid sequence of SEQ ID NO: 1, the GDP-mannose-4,6-dehydratase comprises an amino acid sequence of SEQ ID NO: 5, the GDP-L-fucose-synthase comprises an amino acid sequence of SEQ ID NO: 6, the glucokinase comprises an amino acid sequence of SEQ ID NO: 1, the phosphomannomutase comprises an amino acid sequence of SEQ ID NO: 2, and the N-acetylhexosamine-1-kinase comprises an amino acid sequence of SEQ ID NO: 3.
